(19) United States
(12) Patent Application Publication
(10) Pub. No.: US 2018/0280557 A1
(43) Pub. Date: Oct. 4, 2018

(54) SCENT DISPENSER SUITABLE FOR USE IN ENCLOSED ENVIRONMENTS, FOR INSTANCE FOR USE IN VEHICLES

(71) Applicant: VAPOR COMMUNICATIONS, INC., Cambridge, MA (US)

(72) Inventors: Rachel Diane Field, Huntington Beach, CA (US); David A. Edwards, Cambridge, MA (US); Donald Peter Zereski, Shrewsbury, MA (US)

(21) Appl. No.: 15/765,703

(22) PCT Filed: Oct. 5, 2016
(86) PCT No.: PCT/US2016/055541

§ 371 Date: Apr. 3, 2018

Related U.S. Application Data
(60) Provisional application No. 62/237,304, filed on Oct. 5, 2015.
(60) Provisional application No. 62/314,727, filed on Mar. 29, 2016.
(60) Provisional application No. 62/398,994, filed on Sep. 23, 2016.

(51) Int. Cl. A61L 9/12; B60H 3/00

(52) U.S. Cl. CPC A61L 9/122; B60H 3/0035; B60H 2003/0042; B60H 2003/0057; B60H 2003/0064; A61L 2209/11; A61L 2209/134; A61L 9/125; A61L 2209/133

(57) ABSTRACT

A scent dispenser includes a housing, holder having scent cartridge receiver(s) to removably hold scent cartridges, baffle(s) with a port which is positionable, actuator(s) (e.g., stepper motors), and a control subsystem that drives the actuators to position the baffles to selectively dispense scent from the scent cartridges. The scent cartridges can have passages, at least some of which contain scent media, and optionally one passage without scent media. The housing can be cylindrical and fittable in a cup holder of an automobile. The scent dispenser can have vents at a bottom, a grille at a top, and an air mover (e.g., fan) to create an air flow from the vents through the grille, passing through any scent channels which are aligned with the ports to dispense scent. One or more substrates (e.g., printed circuit boards) can have apertures (e.g., large central passage) to facilitate the air flow.

### BACKGROUND

### Technical Field

This disclosure generally relates to providing olfactory sensations to one or more users, for example users in an enclosed environment, for instance an interior of a vehicle such as an automobile.

### Description of the Related Art

All of our five senses act as messengers that deliver information to the brain, which then processes this information, causing us to respond in relatively predictable ways. Within the context of our sense of smell, all odors present themselves in specific chemical configurations, allowing humans to perceive a wide variety of distinct odors. Odor perception initiates in the nose, where the respective molecules are detected by a large family of olfactory receptors. Olfactory receptors have diverse protein sequences, and are assigned to subfamilies on the basis of sequence relationships. These observations formed the basis for research into the mechanisms underlying human odor perception, leading to the 2004 grant of the Nobel Prize in Physiology and Medicine to Linda B. Buck and Richard Axel.

However, even given the significant importance of our sense of smell, relatively little has been done to develop the apparent physiological value of this sense or to more thoroughly incorporate this sense into how humans experience the world around them on a daily basis. Although some systems and devices have been proposed for attempting to provide olfactory sensations to users, such systems and devices have proven inadequate as mobile, personal, targeted and effective delivery systems that may be used to alter behavior. Some of these systems and devices are intended for personal use, while other systems and devices are intended to provide olfactory sensations simultaneously to groups of people, for example people located in a common area such as a movie theater.

New approaches that selectively automate, including more precisely control and remotely deliver, desired scents, as well as optionally coordinate audio and/or visual stimuli with olfactory sensations in order to deliver a physiological response for personal and/or group use, are desirable.

### BRIEF SUMMARY

A scent dispenser includes a housing, a holder having a number of scent cartridge receivers to removably hold a number of scent cartridges, and a number of baffles or shutters, each with a port, and which are selectively positionable with respect to respective ones of the scent cartridge receivers and, hence, with respect to the scent cartridges held by the scent cartridge receivers to selectively dispense scent from the scent cartridges. The scent dispenser further includes a number of actuators (e.g., stepper motors) and a control subsystem that drives the actuators to position the ports of the baffles to selectively dispense scent from the scent cartridges. The scent cartridges can have a plurality of passages, at least some of which contain scent media. Optionally, one passage can be devoid or without scent media, or alternatively contain a neutral scent media (e.g., non-discernable scent). As a further alternative, one of the positions can be one in which the port of a baffle does not align with any passage of the scent cartridge, thereby preventing dispersion (e.g., molecular diffusion) of scent. The scent dispenser can have inlets or vents at a bottom, a grille at a top, and an air mover (e.g., fan) to create an air flow from the inlets or vents through the grille, passing through any passages of the corresponding scent cartridges that are aligned with the ports to dispense scent from the scent media in the passages.

The scent dispenser may include one or more substrates (e.g., printed circuit boards) in the housing. The substrates can have apertures (e.g., large central passages) to facilitate the air flow from the vents to the top outlet or grille. The substrates can carry various components. For example, one substrate can carry the actuators (e.g., stepper motors). Also for example, one substrate can carry a fan. Also for example, one substrate can carry the control subsystem or portion thereof.

The scent dispenser may include one or more power sources, for example a primary or a secondary battery. The power source can be located in the housing, and, for example, be supported by one or more spacers, spaced from a bottom of the housing and the inlets of vents. The spacers can be thermally conductive (e.g., metal), and serve as heat skins to, for example, draw heat from the power source and/or electronics.

The housing can be cylindrical, and can have a diameter sized to fit in a cup holder, for example a cup holder of a vehicle such as an automobile. Thus, the scent dispenser can serve as a “smart” air freshener for an enclosed environment, for instance an interior of a vehicle. Also for example, the scent dispenser can advantageously release scent(s) in a vehicle to increase alertness while driving, and modulate the amounts and types of scents released to avoids “nose fatigue” for the driver or pilot.

The baffles can, for example, be coupled to actuators (e.g., stepper motors) for rotational movement with respect to respective scent cartridges. The baffles are typically automatically or autonomously moveable in response to signals. In some implementations, the scent dispenser can be operated to deliver scent based on media content (e.g., video content, audio content, audiovisual content) being presented, for instance via a display of a head unit of the vehicle and/or sound system of the vehicle.

The scent dispenser can be communicatively coupled with a source of instructions or commands, for example communicatively coupled with a control subsystem, a terrestrial or satellite broadcaster, or RF or NFC beacons. The communicative coupling can be tethered (i.e., wires, optical fiber, cable(s)). The communicative coupling can be untethered (i.e., radio frequency or microwave frequency transmitters, receivers and/or radios; infrared transmitters and/or receivers).

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn, are not necessarily intended to convey any information regarding the actual shape of the particular elements, and may have been solely selected for ease of recognition in the drawings.

FIG. 1A is a top, front isometric view of a scent dispenser according to at least one illustrated implementation, along with a number of scent cartridges removably mounted in the scent dispenser, each scent cartridge having a plurality of passages, at least some of which contain scent media.

FIG. 1B is a bottom plan view of the scent dispenser of FIG. 1A, better illustrating a plurality of vents or inlet ports that extend about a periphery of the scent dispenser, according to at least one illustrated implementation.

FIG. 1C is a top, front isometric view of the scent dispenser of FIG. 1A, with a shroud or nacelle or collar and a grille removed to better illustrate an interior of the scent dispenser, including a plurality of spacers or steps, according to at least one illustrated implementation.

FIG. 2A is a top, front, right isometric view of the scent dispenser of FIG. 1A, with a housing, base or bottom portion removed to better illustrate various components of the scent dispenser which are housed in the interior of the scent dispenser, according to at least one illustrated implementation.

FIG. 2B is a front, left, top isometric view of various components of the scent dispenser of FIG. 1A, including baffles or shutters with ports and actuators (e.g., stepper motors) coupled to move the baffles to selectively align the ports with scent media bearing passages of respective scent cartridges, according to at least one illustrated implementation.

FIG. 2C is a top plan view of various components of the scent dispenser of FIG. 1A, including the baffles or shutters with ports and a first substrate (e.g., printed circuit board), according to at least one illustrated implementation.

FIG. 2D is a bottom, rear, right, isometric view of various components of the scent dispenser of FIG. 1A, according to at least one illustrated implementation.

FIG. 3A is a top, left, rear isometric view of baffles, stepper motors and a first substrate of the scent dispenser of FIG. 1A, according to at least one illustrated implementation.

FIG. 3B is an elevational view of the baffles, stepper motors and the first substrate of the scent dispenser of FIG. 1A, according to at least one illustrated implementation.

FIG. 4 is a top isometric view of a stepper motor, according to at least one illustrated implementation, illustrating a drive shaft of the stepper motor.

FIG. 5 is bottom isometric view of a baffle with a port, according to at least one illustrated implementation, illustrating a stem to physically couple with the drive shaft of the stepper motor.

FIG. 6A is a top, right, front isometric view of a holder of the scent dispenser of FIG. 1A, the holder having a number of scent cartridge receivers, according to at least one illustrated implementation.

FIG. 6B is a top plan view of the holder of the scent dispenser of FIG. 1A, according to at least one illustrated implementation.

FIG. 6C is a bottom isometric view of the holder of the scent dispenser of FIG. 1A, according to at least one illustrated implementation.

FIG. 6D is a bottom plan view of the holder of the scent dispenser of FIG. 1A, the holder having a lip or apron to closely receive the baffles, according to at least one illustrated implementation.

FIG. 7A is a top isometric view of a scent cartridge for use with the scent dispenser of FIG. 1A, the scent cartridge having a number of passages, at least some of which contain scent media, and optionally having a key to be received via a keyway of the holder of the scent dispenser, according to at least one illustrated implementation.

FIG. 7B is a top plan view of the scent cartridge of FIG. 7A, according to at least one illustrated implementation.

FIG. 7C is a bottom isometric view of the scent cartridge of FIG. 7A, according to at least one illustrated implementation.

FIG. 7D is a bottom plan view of the scent cartridge of FIG. 7A, according to at least one illustrated implementation.

FIG. 8A is a top plan view of the scent dispenser of FIG. 1, showing the baffle in a first or neutral position which, for example, aligns the port or vent with none of the scent media containing passages of the scent cartridge, to prevent dispensing of scent from the scent dispenser.

FIG. 8B is a top plan view of the scent dispenser of FIG. 1, showing the baffle rotated or pivoted into a second position which, for example, aligns the port or vent with a first one of the scent media containing passages of the scent cartridge, to dispense scent of a first fragrance type from the scent dispenser.

FIG. 8C is a top plan view of the scent dispenser of FIG. 1, showing the baffle rotated or pivoted into a third position which, for example, aligns the port or vent with a second one of the scent media containing passages of the scent cartridge, to dispense scent of a second fragrance type from the scent dispenser.

FIG. 8D is a top plan view of the scent dispenser of FIG. 1, showing the baffle rotated or pivoted into a fourth position which, for example, aligns the port or vent with a third one of the scent media containing passages of the scent cartridge, to dispense scent of a third fragrance type from the scent dispenser.

FIG. 8E is a top plan view of the scent dispenser of FIG. 1, showing the baffle rotated or pivoted into a fifth position which, for example, aligns the port or vent with a fourth one of the scent media containing passages of the scent cartridge, to dispense scent of a fourth fragrance type from the scent dispenser.

FIG. 9 is a top isometric view of a plurality of scent cartridges of FIG. 7A and a harness physically coupled to hold the plurality of scent cartridges in a fixed position and orientation with respect to one another according to at least one illustrated implementation.

FIG. 10 is a top plan view of the harness of FIG. 9, showing a plurality of plugs where, for example, for each of the scent cartridges at least one respective plug received through a respective passage to securely fasten a respective scent cartridge to the harness according to at least one illustrated implementation.

FIG. 11 is a side elevational view of a scent cartridge unit which includes a body, a top cover or baffle, a bottom cover or baffle, and a drive interface, coupled together as an integral scent cartridge unit, according to at least one illustrated implementation.

FIG. 12 is a cross-sectional view of the scent cartridge unit of FIG. 11, at, rotated counterclockwise from the illustration of FIG. 11.

FIG. 13A is an isometric view of the body of the scent cartridge unit of FIG. 11.

FIG. 13B is a rear side elevational view of the body of the scent cartridge unit of FIG. 11.

FIG. 13C is a top plan view of the body of the scent cartridge unit of FIG. 11.

FIG. 13D is a cross-sectional view of the body of the scent cartridge unit of FIG. 11.

FIG. 13E is a front elevational view of the body of the scent cartridge unit of FIG. 11.

FIG. 14A is a top isometric view of the top cover or baffle of the scent cartridge unit of FIG. 11.

FIG. 14B is a bottom isometric view of the top cover or baffle of the scent cartridge unit of FIG. 11.

FIG. 14C is a cross-sectional view of the top cover or baffle of the scent cartridge unit of FIG. 11.

FIG. 14D is a top plan view of the top cover or baffle of the scent cartridge unit of FIG. 11.

FIG. 14E is a side elevational view of the top cover or baffle of the scent cartridge unit of FIG. 11.

FIG. 14F is a bottom plan view of the top cover or baffle of the scent cartridge unit of FIG. 11.

FIG. 15A is a top plan view of the bottom cover or baffle of the scent cartridge unit of FIG. 11.

FIG. 15B is a side elevational view of the bottom cover or baffle of the scent cartridge unit of FIG. 11.

FIG. 15C is a bottom plan view of the bottom cover or baffle of the scent cartridge unit of FIG. 11.

FIG. 15D is a cross-sectional view of the bottom cover or baffle of the scent cartridge unit of FIG. 11.

FIG. 16A is a top isometric view of the drive interface of the scent cartridge unit of FIG. 11.

FIG. 16B is a bottom isometric view of the drive interface of the scent cartridge unit of FIG. 11.

FIG. 16C is a top plan view of the drive interface of the scent cartridge unit of FIG. 11.

FIG. 16D is a bottom plan view of the drive interface of the scent cartridge unit of FIG. 11.

FIG. 16E is a side elevational view of the drive interface of the scent cartridge unit of FIG. 11.

FIG. 16F is a cross-sectional view of the drive interface of the scent cartridge unit of FIG. 11.

FIG. 17A is a top isometric view of a harness for retaining three of the scent cartridge units of FIG. 11, according to at least one illustrated implementation.

FIG. 17B is a top plan view of the harness of FIG. 17A.

FIG. 17C is a bottom plan view of the harness of FIG. 17A.

FIG. 17D is a side elevational view of the harness of FIG. 17A.

FIG. 17E is a cross sectional view of the harness of FIG. 17A.

FIG. 17F is a detailed view of a portion of the harness of FIG. 17A.

FIG. 17G is a detailed view of a portion of the harness of FIG. 17A.

### DETAILED DESCRIPTION

In the following description, certain specific details are set forth in order to provide a thorough understanding of various disclosed implementations. However, one skilled in the relevant art will recognize that implementations may be practiced without one or more of these specific details, or with other methods, components, materials, etc. In other instances, well-known structures associated with communications systems or interfaces, computer systems, server computers, and/or communications networks have not been shown or described in detail to avoid unnecessarily obscuring descriptions of the implementations.

Unless the context requires otherwise, throughout the specification and claims that follow, the word “comprising” is synonymous with “including,” and is inclusive or open-ended (i.e., does not exclude additional, unrecited elements or method acts).

Reference throughout this specification to “one implementation” or “an implementation” means that a particular feature, structure or characteristic described in connection with the implementation is included in at least one implementation. Thus, the appearances of the phrases “in one implementation” or “in an implementation” in various places throughout this specification are not necessarily all referring to the same implementation. Furthermore, the particular features, structures, or characteristics may be combined in any suitable manner in one or more implementations.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the implementations.

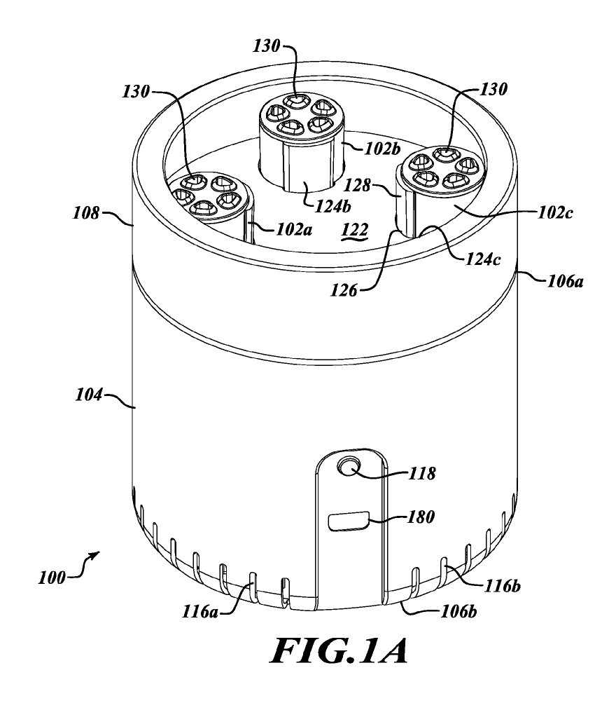

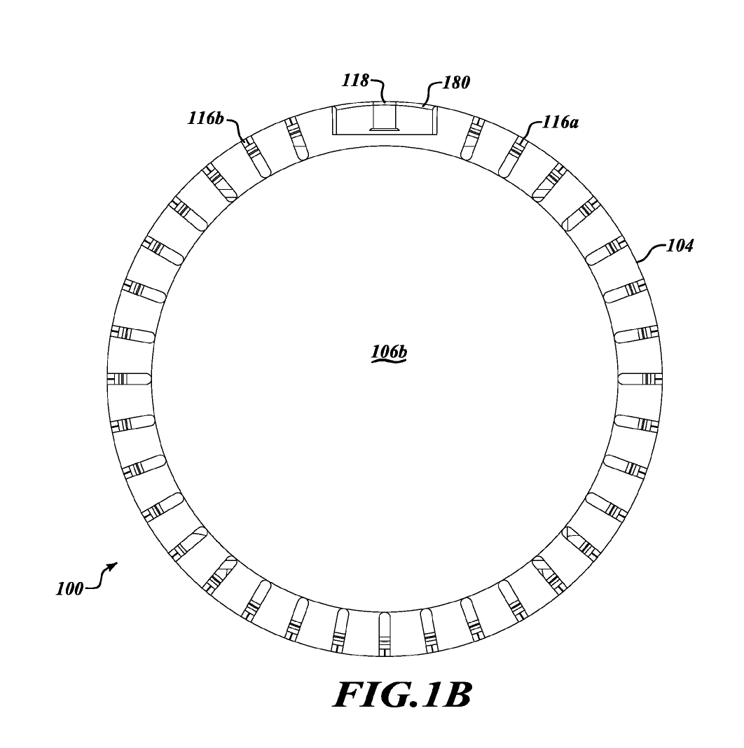

FIGS. 1A-1C show a scent dispenser 100, according to at least one illustrated implementation, which removably holds a number of scent cartridges 102a, 102b, 102c (three shown, collectively 102).

The scent dispenser 100 comprises a housing (also referred to as a base or bottom portion) 104 having a first or upper end 106a, a second or bottom end 106b opposed to the first end 106a, along a length of the housing 104. The housing 104 can be cylindrical, as illustrated, and in some implementations preferably has a diameter at least approximately equal to that of a coffee cup (i.e., 4.5 inches or 114.3 millimeters to 5.75 inches or 146.0 millimeters, plus or minus 10 percent). The housing 104 can be 4.8 inches or 122.7 millimeters in height. Alternatively, the housing 104 can have other shapes of “footprints,” for instance square, rectangular, hexagonal, octagonal or other polygonal forms. The housing 104 can be formed of one or more of a large variety of materials. For example, the housing 104 can be formed of one or more plastics (e.g., thermoplastics, ABS, nylon, PET, polypropylene, Styrene, TPE), and/or alternatively formed of a metal (e.g., aluminum). The housing 104 can be manufactured, for instance via injection molding of plastic or by an additive printing process. The housing 104 can optionally be formed of a pliable and/or resilient material. The housing 104 can optionally include a resilient silicone sleeve.

The scent dispenser 100 can include a shroud or nacelle (also referred to as a collar) 108, for instance removably physically coupled to the housing 104 at the first end or upper end 106a thereof. The shroud or nacelle 108 can, for example, be press fit to the housing 104, or removably attached thereto via a pair of mating screw threads (not shown), bayonet type mount (slots and tabs, not shown) or via fasteners (not shown). The shroud or nacelle 108 can be cylindrical, as illustrated, with a diameter that at least approximately matches a diameter of the housing 104. Alternatively, the shroud or nacelle 108 can have other shapes of “footprints” for instance square, rectangular, hexagonal, octagonal or other polygonal forms. The shroud or nacelle 108 can be formed of one or more of a large variety of materials. For example, the shroud or nacelle 108 is preferably formed of a metal (e.g., aluminum, stainless steel) and is washable, to avoid being tainted by the dispersed scents. Less preferably, the shroud or nacelle 108 can be formed from one or more plastics (e.g., thermoplastics, ABS, Nylon, PET, polypropylene, Styrene, TPE). The shroud or nacelle 108 can be formed or manufactured, for instance via extrusion or stamped forming, or alternatively by injection molding of plastic or by an additive printing process.

As best illustrated in FIG. 1C, the housing 104 has at least one wall 110 that separates an interior 112 of the housing 104 from an exterior 114 thereof. The housing 104 includes a plurality of inlets or vents 116a, 116b (only two called out, collectively 116) that allow passage of air to the interior 112 of the housing 104 from the exterior 114 thereof. The inlets or vents 116 can be positioned at least proximate a second or bottom end 106b of the housing 104.

As visible in FIG. 1A, the housing 104 optionally includes a user input element or control, for example a switch 118, selectively operable to turn the scent dispenser ON and OFF. The switch 118 is accessible from the exterior 114 of the housing 104. As is also visible in FIG. 1A, the housing 104 optionally includes a user output element (not shown), for example a light (e.g., LED) or display (e.g., LCD) to provide information to a user, for instance operational status (e.g., ON or OFF), fan speed (e.g., high, medium, low), condition of scent media (e.g., replace, percentage of useful life left), etc. The scent dispenser 100 comprises a holder 122 located in the housing 104. The holder 122 has a number of scent cartridge receivers 124a, 124b, 124c (only two visible in FIG. 1A, collectively 124). The scent cartridge receivers 124 are sized and dimensioned to each removably hold a respective scent cartridge 102. The scent cartridge receivers 124 are designed to substantially prevent lateral movement (e.g., rotation, lateral translation) of the scent cartridges 102 during operation of the scent dispenser 100, yet allow the scent cartridge 102 to be withdrawn (i.e., axial movement or axial translation). Thus, each scent cartridge receiver 124 can include a keyway 126 (only one called out for drawing legibility), the keyway 126 sized and dimensioned to securely engage a key 128 (only one called out for drawing legibility) on the respective scent cartridges 102. Notably, the scent cartridges 102 each have a plurality of passages 130 (only one called out for each scent cartridge 102 in FIG. 1A for drawing legibility). At least one, and typically many, of the passages contain respective scent media which emit (e.g., diffuse) respective scents when exposed to a passage of air or some implementations other stimuli.

As illustrated, the scent cartridge receivers 124 each comprise a respective receptacle, sized and dimensioned to hold respective ones of the plurality of scent cartridges 102 and restrain the respective scent cartridges 102 from movement (e.g. rotation). As illustrated, each of the receptacles has a keyway 126, the keyways 126 sized and dimensioned to securely receive a key 128 on each of the scent cartridges 102 to restrain the respective scent cartridges 102 from movement (e.g., rotation). Alternatively, each of the receptacles can have a key, the key sized and dimensioned to be securely received via a keyway on a respective one of the scent cartridges 102, to restrain the respective scent cartridges 102 from movement (e.g., rotation).

The scent dispenser 100 can include a number of steps or spacers 132a, 132n (only two called out in FIG. 1C, collectively 132) in the interior 112 thereof. The steps or spacers 132 advantageously space various components (e.g., battery) away from the inlets or vents 116. The steps or spacers 132 are preferably thermally conductive (e.g., metal), and serve as heat skins to, for example, draw heat from a power source and/or electronics.

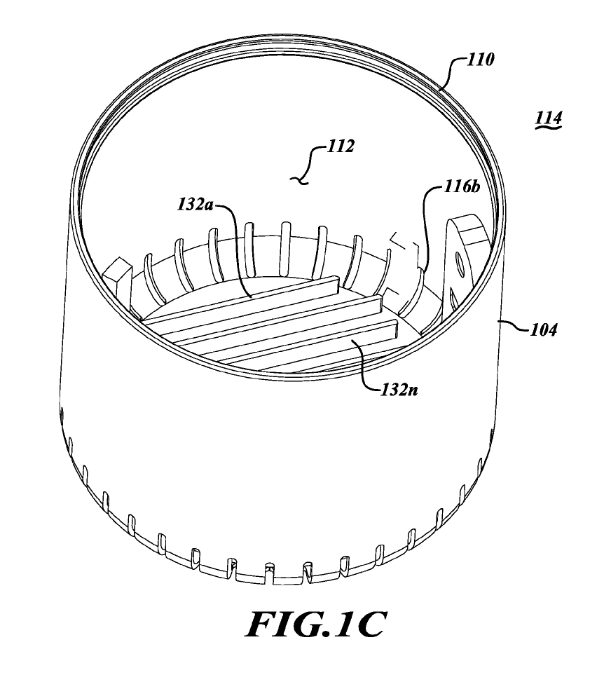

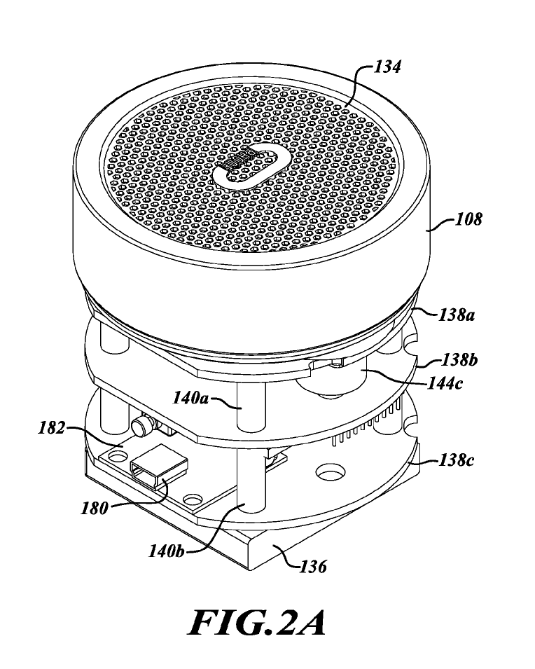

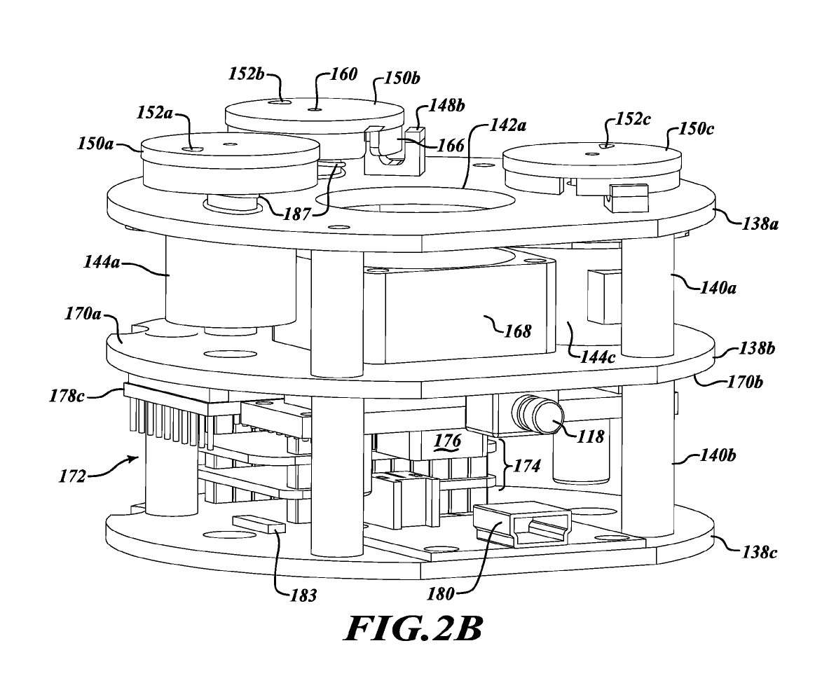

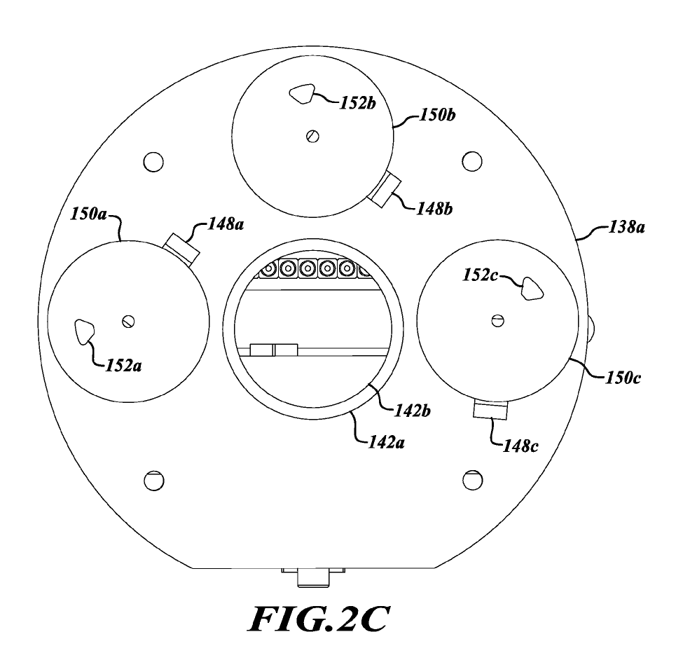

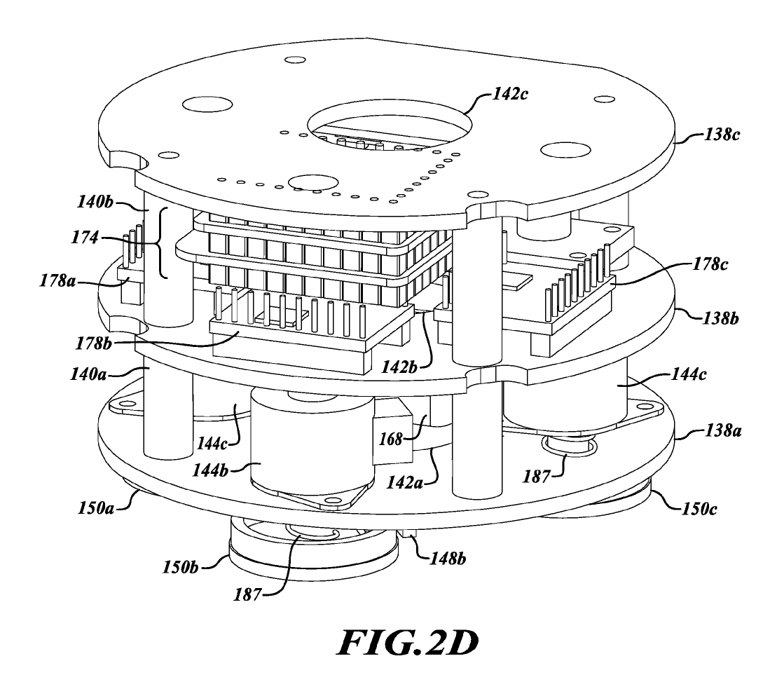

FIG. 2A shows the scent dispenser 100 with the housing 104 removed and a grille 134 located in the shroud or nacelle 108.

The grille 134 can advantageously protect the scent dispenser 100 from foreign debris, as well as providing an aesthetically pleasing visual treatment. The grille 134 can be an integrally structure with the shroud or nacelle 108, or can be separable therefrom to allow a user to change the grille, for example to achieve different aesthetic affects. The shroud or nacelle 108 and grille 134 can be selectively detachable from the housing 104, for example to allow replenishment or replacement of spent scent cartridges 102 with new scent cartridges 102, or to allow different scent cartridges with different scents to be loaded in anticipation of a particular olfactory experience.

As visible in FIG. 2A, the scent dispenser 100 can include one or more power sources 136 positioned in the housing 104. The power source(s) 136 can, as illustrated, take the form of either one or more chemical batteries, for example one or more primary disposable) chemical battery cells (e.g. zinc-carbon, alkaline) or one or more secondary (i.e., rechargeable) chemical battery cells (e.g., nickel-cadmium, nickel-zinc, nickel metal hydride, lithium ion or lithium polymer battery cells). The power source(s) 136 should provide an appropriate voltage and current suitable for the control subsystem, electric stepper motors, and fan. Additionally or alternatively, the scent dispenser 100 can include an array of ultracapacitor cells and/or a source of hydrogen and an array of fuel cells. The steps or spacers 132 can advantageously space the battery 136 away from the inlets or vents 106 to facilitate air flow through the housing 104.

As visible in FIGS. 2A-2D, the scent dispenser 100 can include a number of substrates 138a, 138b, 138c (three shown, collectively 138) positioned in the interior 112 of the housing 104, for example a first substrate 138a, a second substrate 138b and a third substrate 138c. The substrates 138 can, for example, be arranged successively from one another along a longitudinal axis of the housing 104. Substrates 138 can, for example, be spaced from one another by pillars 140a, 140b (only two called out, in FIG. 2A, collectively 140). Substrates 138 can, for example, take the form of circuit boards, for instance printed circuit boards formed of suitable dielectric material (e.g., FR4) with one or more electrically conductive traces printed thereon or on a surface therein. The substrates 138 can be identical or similar to one another in material and construction, or in some implementations can differ from one another. For instance, one of the substrates 138 can take the form of an FR4 material while another one of the substrates 138 can take the form of a direct bonded copper substrate to function as a heat sink. The substrates 138 each have a relatively large aperture or passage 142a, 142b, 142c (e.g., central aperture or passage, collectively 142), to allow passage of air from the inlets or vents 106 through the grille 134 via one or more passages 130 of the scent cartridges 102. Where the housing 104 is cylindrical, a portion of a perimeter of the substrates 138 can be circular, and the substrates 138 thus described can be annular or at least somewhat annular.

The substrates 138 carry various components of the scent dispenser 100.

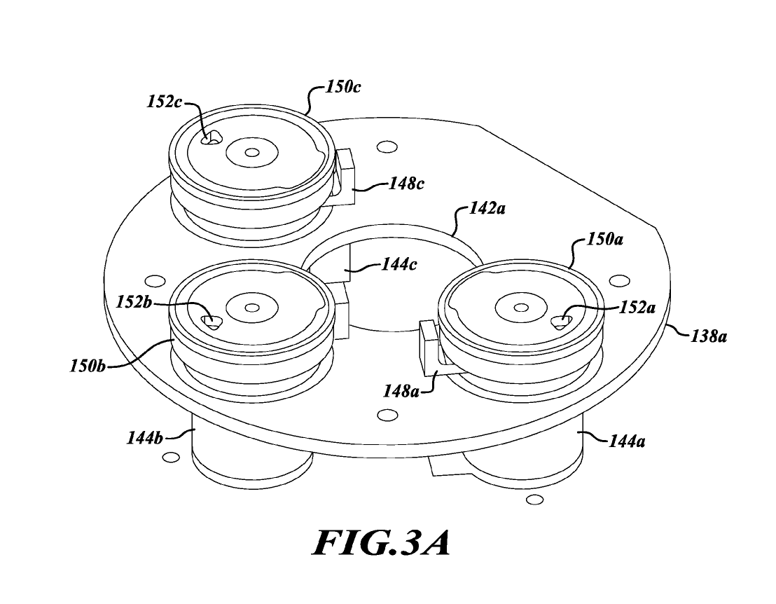

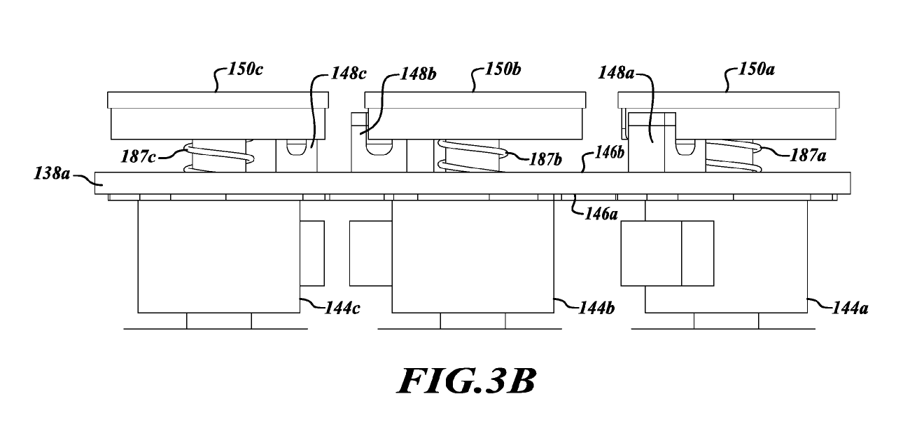

For example, the first substrate 138a can carry a number of actuators, for example stepper electric motors 144a, 144b, 144c (three shown, collectively 144). The stepper electric motors 144 can be attached (e.g., via fasteners, adhesives) to an inner surface 146a (called out in FIG. 3B) of the first substrate 138a, opposed to an outer surface 146b (called out in FIG. 3B) of the first substrate 138a across a thickness of the first substrate 138a and opposed across the first substrate 138a from the holder 122. The first substrate 138a can carry a number of position encoders (e.g., rotary encoder) 148a, 148b, 148c (best illustrated in FIG. 2C, collectively 148). For example, the first substrate 138a can, as illustrated, carry a number of optical position encoders 148, for instance comprised of pairs of a light source (e.g., LED) and an optical sensor (e.g., photodiode) or an optical sensor and a marking. For example, the first substrate 138a can carry a number of magnetic position encoders, for instance comprised of pairs of magnets and magnetic field sensors (e.g., Reed switch). The use of the position encoders 148 are described elsewhere herein.

As best illustrated in FIGS. 2B-2D, the stepper motors 144 are coupled to drive respective ones of a number of baffle 150a, 150b, 150c (three illustrated, collectively 150, also referred to as shutters). Each baffle 150 has at least one respective port 152a, 152b, 152c (three illustrated, collectively 152). Typically, each baffle 150 has a single port 152. The baffles 150 selective open and close a flow of air through the passages 130 of the scent cartridges 102. The baffles 150 are mounted for movement with respect to the at least one scent cartridge receiver 124 of the holder 122, between a plurality of positions, for example, which align with respective passages 130 or align with none of the passages 130 of the scent cartridge 102.

The stepper motors 144 are controlled to drive the respective baffles 150 and/or ports 152 into a set of positions. For example, a first or default one of the positions may provide communicative coupling between none of the passages 130 of the respective port 152 of the respective baffle 150, to prevent dispersion of scent from the interior 112 of the scent dispenser 100 to the exterior 114 thereof. Also for example, a second one of the positions may provide communicative coupling between a first one of the passages 130 of the respective scent cartridge 102 mounted in the respective scent cartridge receiver 124 and the port 152 of the respective baffle 150. Also for example, a third one of the positions may provide communicative coupling between a second one of the passages 130 of the respective scent cartridge 102 mounted in the respective scent cartridge receiver 124 and the port 152 of the respective baffle 150. As a further example, a fourth one of the positions may provide communicative coupling between a third one of the passages 130 of the respective scent cartridge 102 mounted in the respective scent cartridge receiver 124 and the port 152 of the respective baffle 150. Alternatively to the first or default position, a fifth one of the positions may provide communicative coupling between a fourth one of the passages 130 of the respective scent cartridge 102 mounted in the respective scent cartridge receiver 124 and the port 152 of the respective baffle 150, where the fourth one of the passages 130 does not contain any scent media or contains a neutral or neutralizing scent media (e.g., baking soda).

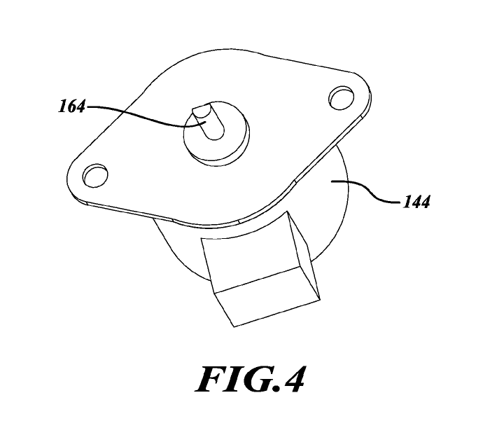

FIGS. 2B, 2D, 3A, 3B, 4 and 5, illustrate the baffles 150 as circular plates 154 (only called out in FIG. 5), each with a circular peripheral edge 156 (only called out in FIG. 5) and a pedestal or leg 158 (only called out in FIG. 5) extending therefrom. The pedestal or leg 158 extends perpendicularly from the plate 154, and is typically aligned with a center 160 (only called out in FIG. 2B) of the circular plate 154. The pedestal or leg 158 has a slot 162 that is sized and dimensioned to securely receive (e.g., slip fit) a drive shaft 164 (only called out in FIG. 4) of a respective stepper motor 144. The slot 162 can, for example have a D-shaped profile, and at least a portion of the drive shaft 164 can have a complementary D-shaped profile. Thus, the drive shaft 164 is drivingly coupled to the one baffle 150, for example to rotate the baffle 150. In some implementations the baffle 150 can include a tab or ear 166 (FIGS. 2B and 5) which extends from the circular plate 154. Passage of the tab or ear 166 is detectable by the respective position encoder 148 (FIGS. 2B, 3A).

In some implementations, stepper motor 144 can rotate the baffle 150 in a clockwise rotation at one time and in a counterclockwise rotation at another time. In some implantations, a solenoid or other actuator can be employed to either laterally (i.e., in plane) translate or rotate a baffle or shutter. In such implementations, the baffle or shutter can have a non-circular profile, for example a rectangular profile, and for example be slideably mounted to translate with respect to a scent cartridge receiver 124.

Also for example, the second substrate 138b can carry an air mover 168, for example illustrated as a fan (e.g., blades and electric motor). Alternatively, the air mover 168 can take the form of a pulse jet air mover. The air mover 168 can be attached to an outer or upper surface 170a (called out in FIG. 2B) of the second substrate 138b, spaced relatively toward the holder 122 with respect to an inner surface 170b (called out in FIG. 2B) of the second substrate 138. The air mover (e.g., fan) 168 can be disposed in or encompass the relatively large air flow aperture or passage 142b of the second substrate 138b. Otherwise, the relatively large air flow apertures or passages 142a, 142b, 142c of the first, second and third substrates 138a, 138b, 138c should remain substantially un-occluded.

Also for example, the second substrate 138b can carry a number of motor controllers 178a, 178b, 178c (three shown in FIG. 2D, collectively 178). The motor controllers 178 can be attached to the inner or lower surface 170b (called out in FIG. 2B) of the second substrate 138b, and communicatively controlling coupled to respective ones of the stepper motors 144.

As a further example, the third substrate 138c can carry a control subsystem 172, comprising circuitry including processor(s), microcontroller(s), motor controllers, memory, and communications components. The control subsystem 172 comprises circuitry communicatively coupled to, and operable to control, the at least one actuator (e.g., stepper motor 144) to selectively move (e.g., rotate, translate) the at least one baffle 150 with respect to the at least one scent cartridge receiver 124 between the plurality of positions (e.g., rotational positions or orientations which can be represented in degrees, radians or in hours such as Twelve O'clock, Five O'clock, etc.).

The control subsystem 172 includes control circuitry, for example a microcontroller board 176 and motor controllers 178.

A microcontroller board (e.g., Mini DUINO®) 174 may include a microcontroller 176 having logic circuitry, nontransitory nonvolatile memory (e.g., ROM, FLASH, EEPROM), and nontransitory volatile memory (e.g., RAM, registers). A suitable microcontroller 176 may take the form of an 8-bit microcontroller with in-system programmable flash memory, such as the microcontroller commercially available from Atmel Corporation under designation ATMEGA48/88/168-AU. The microcontroller 176 executes a program stored in its memory, and sends signals to control the various other components, for instance the activation elements, the stepper motors 144, fans 168, optional user interface, optional wired or wireless communications ports, transmitters, receivers, transceivers (collectively radios), etc. Control signals may take on any of a large variety of forms, including analog or digital signals or pulse width modulated (PWM) signals. For instance, the microcontroller 176 may control the air mover (e.g., fan) 168 simply by completing a circuit that powers the air mover 168. For instance, the microcontroller 176 may provide digital control signals to motor controllers 178 to control the stepper motors 144.

While generally discussed as a microcontroller, the control subsystem 172 can optionally include one or more controllers, microcontrollers, processors, microprocessors, digital signal processor (DSPs), graphical processing units (GPUs), and/or application-specific integrated circuits (ASICs). The control subsystem 172 can optionally include one or more nontransitory computer- or processor-readable medium. The nontransitory computer- or processor-readable medium may, for example include one or more nonvolatile memory (e.g., read-only memory (ROM), Flash memory), spinning disc memory (e.g., magnetic disk, optical disk). The nontransitory computer- or processor-readable medium may, for example include one or more volatile memory (e.g., random access memory (RAM)). In some implementations, the nontransitory computer- or processor-readable medium stores at least one of processor executable instructions and/or data, which when executed by the at least one processor causes the at least one processor to control the electric stepper motor 144, for example as discussed elsewhere herein.

The circuitry can include one or more integrated circuit chips such as, for example, various types of processors (e.g., microcontrollers, microprocessors, digital signal processors), drive circuits, nontransitory processor-readable storage media (e.g., one or more nontransitory storage media, for instance nonvolatile memory (e.g., ROM, FLASH) and volatile memory (e.g., RAM)), a communications subsystem including wireless receiver (e.g., radio) signal processing hardware, and decryption hardware as described below in more detail. The circuitry can take the form of a custom system-on-chip (SOC).

The microcontroller 176 is communicatively coupled to receive positional information from the position sensors or position encoders 166. The previously described position encoders 166 (FIG. 2B, 2C, 3A, 3B, 5) can be used to detect a position of respective ones of the baffles 150 or, in particular, the ports 152 of the baffles 150. For example, an optical or magnetic rotary encoder 166 can detect information representative of a rotational position of the baffles 150. As illustrated, an optical encoder 166 can optically detect a passage of a portion (e.g., tab or ear 166, FIGS. 2B, 5) of the baffle 150, for instance by detecting an interruption in light. Also as illustrated, the tab or ear 166 can be positioned diametrically opposed to the port 152 of the baffle 150. The microcontroller 176 can determine or confirm the position of the port 152 of any of the baffles 150 from positional information received from the position sensors or position encoders 166.

The motor controllers 178 are communicatively coupled to control respective ones of the stepper motors 144. The motor controllers 178 are communicatively coupled to the microcontroller 176 for control thereby. The motor controllers 178 include logic circuitry, which can be mounted on a substrate or pedestal and can include one or more heat transfer devices (e.g., fins, pins) which provide a relatively larger surface area to more effectively convectively transfer heat from the logic circuitry. The logic circuitry translates commands from the microcontroller 172 to drive signals to drive the respective actuator, for example to cause the stepper motor 144 to step a defined number of steps.

The scent dispenser 100 may include one or more receivers or ports 180 (one illustrated) to receive communications and/or power. For example, the scent dispenser 100 can include a USB compliant port, to receive both communications and electrical power. The port 180 is accessible from the exterior 114 of the housing 104. The port 180 can advantageously facilitate communicative coupling between the scent dispenser 100 and an external source of signals or information, for example via one or more wires, ribbon cables, optical fibers, or cables. Such can be used to provide control signals from the external source to the scent dispenser 100 to control operation of the scent dispenser 100. Such can additionally or alternatively be used to provide power (e.g., electrical power) from an external power source to the scent dispenser 100.

The control subsystem 172 can include a port or receiver or connector or receptacle to receive control signals or other input. For example, the control subsystem 172 can include a wired port or wired receiver (e.g., Ethernet®, USB®, Thunderbolt®, Lighting®, electrical or optical signaling) to receive signals from an external source. Also for example, the control subsystem 172 can include a wireless port or wireless receiver (e.g., receiver, transceiver, radio, 802.11 compliant, BLUETOOTH®, WI-FI®, radio frequency, microwave frequency or infrared signaling) to wirelessly receive signals from an external source (e.g., smartphone, tablet computer, server computer, other processor-based device). For instance, a BLUETOOTH® compliant radio can provide short-range wireless communications therebetween. The control subsystem 172 can include one or more antennas (e.g., stripline RF antenna) for wireless communications.

The scent dispenser 100 can include recharger circuitry (i.e., recharger) and/or power supply circuitry (e.g., step up DC/DC power converter). The recharger is electrically coupled to receive a supply of electrical power from the port 180, and operable to control a recharging of the battery 136 via an appropriate charging algorithm. Port 180 can be coupled to a USB card 182 which include circuitry and logic to read USB compliant drives or communications. In some implementations, the microcontroller 176 may, for example, control the recharger via control signals, for instance pulse width modulated (PWM) control signal. In some implementations, power supply circuitry may step up a voltage (e.g., up to 12 Volts) from the battery 136 to power the stepper motors 140.

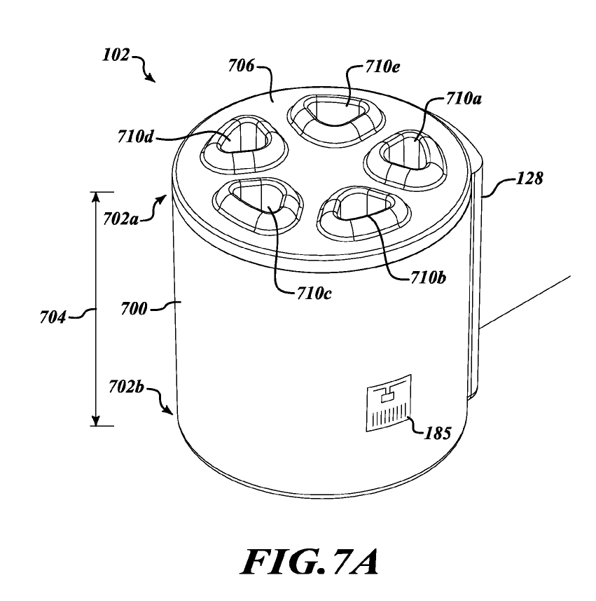

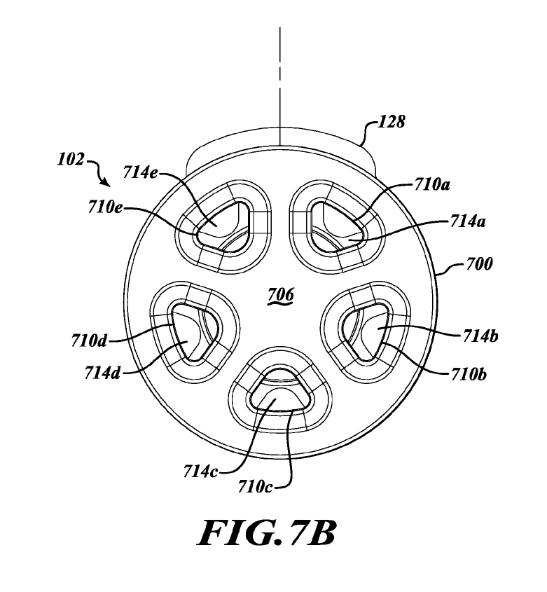

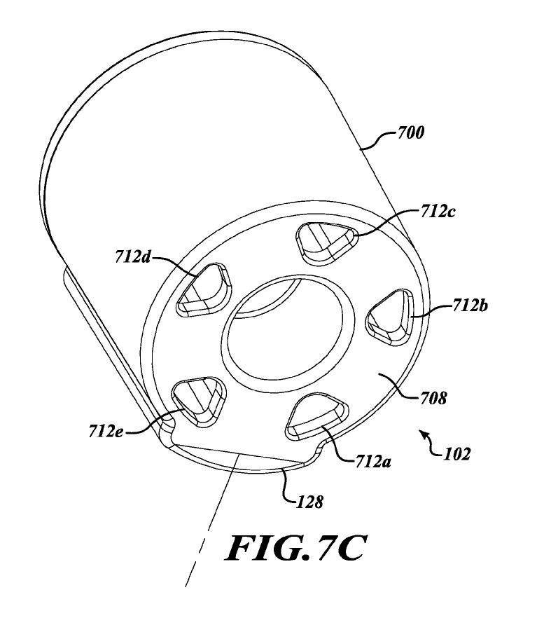

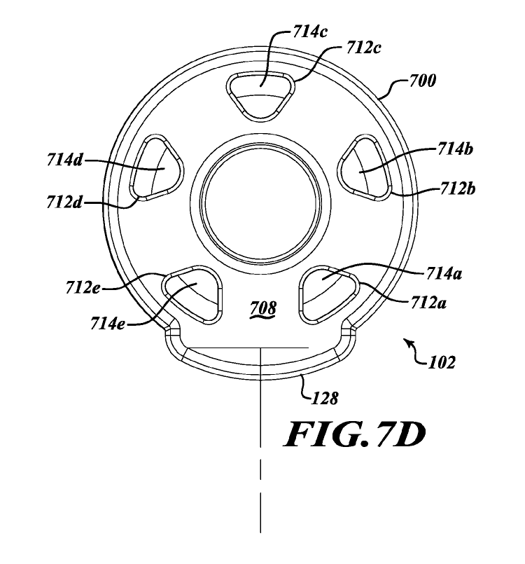

The control subsystem 172 can optionally include a transducer or reader 183 (FIG. 2B) to read machine-readable information (e.g., contents indicator) from the scent cartridges 102. The transducer or reader 183 reads or otherwise senses information from a respective machine readable ID structure 185 (Figures7A). The machine readable ID structure 185 (FIG. 7A showing a combination RFID transponder and optical machine-readable symbol data carrier carried by a scent cartridge 102) can, for example comprise one or more of a wireless transponder/RFID tag, a label with optically machine-readable symbol or text, a magnetic strip. Respective machine readable ID structure 185 are physically associated with each of the scent cartridges 100.

The reader 183 can, for example, take the form of a radio frequency identification (RFID) reader or interrogator. The RFID reader or interrogator can wirelessly read information stored in a respective RFID transponder or tag, physically coupled to attached to each of the scent cartridges 102. If the scent cartridge 102 bears a machine-readable symbol (e.g., barcode symbol, matrix code symbol, picture, text), the reader 183 can take the form of an optical machine-readable symbol reader (e.g., scanning laser type, imager type) to optically read the encoded information. If the scent cartridge 102 bears a magnetic stripe, the reader 183 can take the form of a magnetic stripe reader to read magnetically read information encoded in the magnetic stripe. In each instance, the reader 183 can be incorporated into the housing 104 of the scent dispenser 100, or alternatively be provided separately therefrom, for example tethered to the housing 104 as a distinct unit.

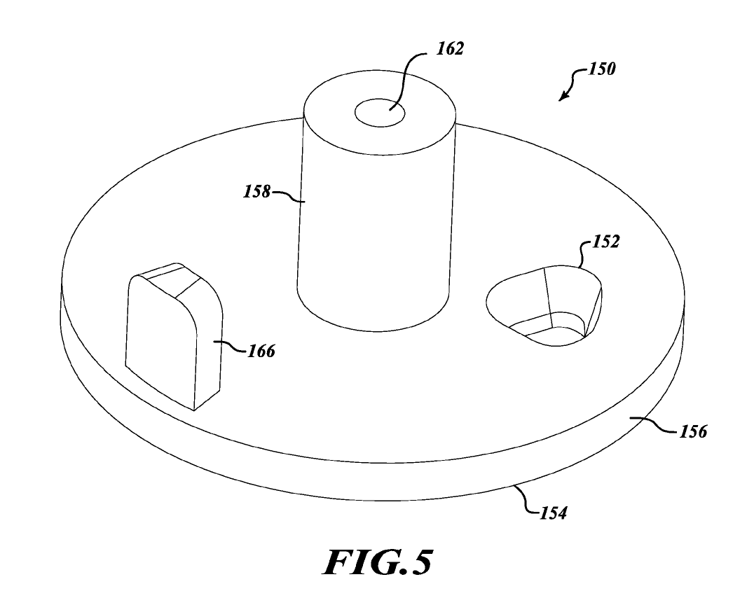

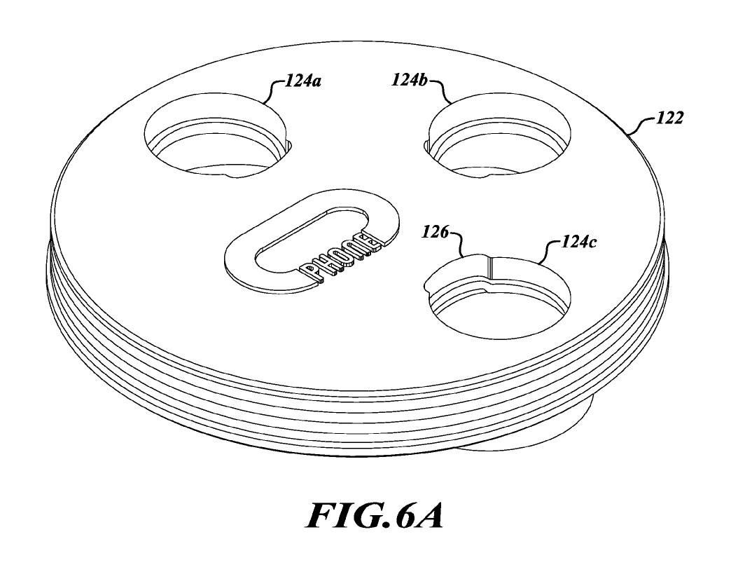

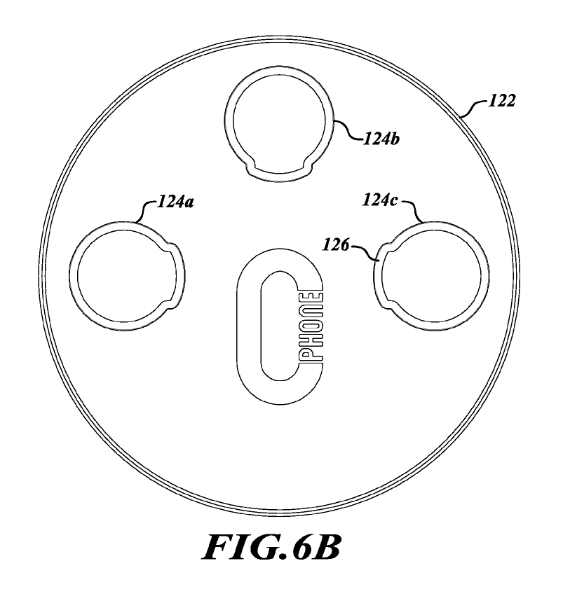

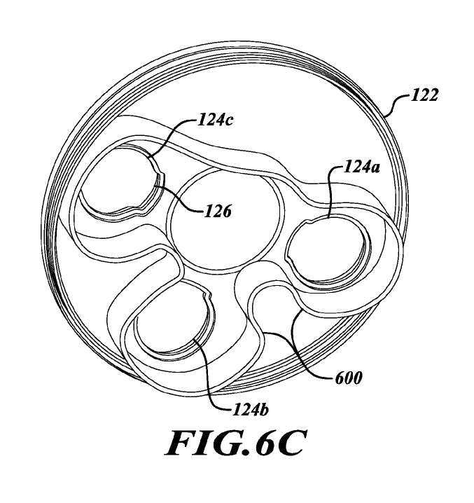

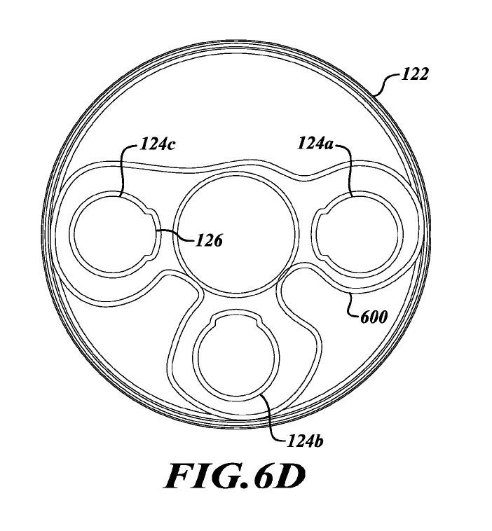

While generally not illustrated, the scent dispenser 100 may include a number of user input/output (I/O) elements or mechanisms. For example, the scent dispenser 100 may include a touchscreen and/or a number of user operable switches, buttons or keys. In some implementations, the operation of the scent dispenser 100 is selectively controllable by a user through the user interface, for example a remote control or an external processor-based device. Switches, buttons or keys may include a power or ON/OFF switch, button or key 118, a volume or sound decrease switch, button or key, and a volume or sound increase switch, button or key. Switches, buttons or keys may include a stop switch, button or key, a pause switch, button or key, a rewind switch, button or key and/or a fast forward switch, button or key, for example. FIGS. 6A-6D show an exemplary holder 122, according to at least one illustrated embodiment.

The control subsystem 172 can be responsive to a state of switch 118. Operation or activation of the switch 118 can toggle the control system between ON and OFF states or conditions.

As previously noted, the holder 122 includes a plurality of scent cartridge receivers, for example in the form of receptacles 124 sized and dimensioned to each removably hold a respective scent cartridge 102 and substantially prevent lateral (i.e., in plane) movement (e.g., rotation, lateral translation) of the scent cartridges 102 during operation of the scent dispenser 100, yet allow the scent cartridge 102 to be withdrawn (i.e., axial movement or axial translation). Thus, each scent cartridge receiver 124 can include a keyway 126 (only one called out for drawing legibility), the keyway 126 sized and dimensioned to securely engage a key 128 (only one called out for drawing legibility) on the respective scent cartridges 102. The scent cartridges 102 are inserted into the receptacles 124 from an upper side or surface of the holder 122.

As best illustrated in FIGS. 6C and 6D, the holder 122 preferably includes at least one lip or skirt 600, which extends from a bottom side or surface (i.e., surface or side on which the baffles 150 reside) of the holder 122. The lip(s) or skirt(s) 600 is/are sized and dimensioned to closely receive a periphery (e.g., circular peripheral edge 156) of the baffles 150. The lip(s) or skirt(s) 600 is/are preferably formed of a resilient or conforming material (e.g., silicone rubber). The lip(s) or skirt(s) 600 help ensure that a path of the airflow is through the passages 142 of the scent cartridges, rather than bypassing the scent cartridges.

Thus, the scent cartridges 102 and baffles 150 all partially sit in or partially reside in the holder 122. The holder 122 thus separates the interior 112 of the housing 104 from the exterior 114, ensuring that the airflow is directed outward of the interior 112 via any passages 130 of the scent cartridges 102 which are aligned with ports 152 of respective baffles 150.

As best illustrated in FIG. 3B, the scent dispenser 100 can include a number of springs 187a, 187b, 187c (three shown in FIG. 3B, collectively 187) that bias respective ones of the baffles 150 into contact with a bottom portion of a respective one of the scent media cartridges 102. The springs 187 can be positioned between a portion of the electric stepper motor 144 and the respective baffle 150.

FIGS. 7A-7D show an exemplary scent cartridge 102, according to at least one illustrated embodiment.

The scent cartridge 102 has a body 700, with a first end 702a and a second end 702b, opposed across a length 704 of the body 700 from the first end 702a. The body 700 forms an interior separated by at least one wall from an exterior of the scent cartridge 102. A portion of the body 700 can form a key 128, to be received by a keyway 126 (FIG. 1A) of a scent cartridge receiver 124. The body 700 can, for example, be generally cylindrical, preferably with a key 128 or keyway to match a complementary structure (e.g., complementary keyway or key) of the scent cartridge receiver 124 and thereby prevent the scent cartridge 102 from rotating with the baffle 150.

The scent cartridge 102 has a first cap or first cover 706 that covers the first end 702a and a second cap or second cover 708 that covers the second end 702b. The first cap or first cover 706 has a plurality of openings 710a-710e (five shown, collectively 710), and the second cap or second cover 708 has a plurality of openings 712a-712e (five shown, collectively 712). Typically, the number of openings 712 in the second cap or second cover 708 will equal the number of openings 710 in the first cap or first cover 706. A number distinct passages 714a-714e (five shown, collectively 714) extend between the openings 710 in the first cap or cover 706 and respective ones of the openings 712 in the second cap or cover 708. At least one of the passages 714 contains or holds scent media. Typically, all of the passages 714 will contain or hold scent media, and the scent cartridges 102 can have a rotational position in which there is no opening or passage aligned with the port 152 of a respective baffle 150, thereby preventing dispersion of scent. Alternatively, one or more scent cartridges 102 can have a passage 714 that is devoid of scent media. Alignment of the port 152 with this “empty” position prevents dispensing of scent from the scent dispenser 100. Alternatively, one or more scent cartridges can have a passage that includes a neutral or neutralizing scent media (e.g., baking soda), that is one with a scent that is not discernable to humans. Alignment of the port 152 with this “neutral” position can dispense a neutral (i.e., undiscernible scent) or can neutralize existing scents. In some implementations, the scent media in the passages 714 is capable of releasing a respective distinct scent. The scent media may, for example, take the form of a powder or a wax (e.g., paraffin wax substrate impregnated with at least one volatile scent material) bearing fragrance. The scent media is typically consumable, and so is replaceable in the scent dispenser 100. Various scent media are discussed in U.S. patent application Ser. No. 14/213,608, filed Mar. 14, 2014; Ser. No. 14/213,683, filed Mar. 14, 2014; 61/944,870, filed Feb. 26, 2014; and 62/199,763, filed Jul. 31, 2015, each of which are incorporated by reference herein in their entireties.

The scent cartridges 102 can be replaceable, for example replaceable scent cartridges. This advantageously allows replenishment of spent scent cartridges 102 or replacement of scent cartridges 102 based on a set of scents that are desired to be available. This may be particularly useful, allowing a set of scents to be loaded based on a theme of a desired experience (e.g., jungle scents, animal scents, food or wine scents, floral scents, urban scents, warfare related scents). Scent media cartridges with various scents can be packaged in sets or collections, for example based on themes.

As previously explained, each scent cartridge 102 can carry (e.g., enclosed, bear, have attached) a respective machine readable ID structure 185, which is readable by a reader 183 to, for example, determine which scents the cartridge can emit or release. Applicants anticipate that any one of such exemplary types of machine-readable ID structure 185 and/or machine-readable identification formats and associated reading hardware may become a standard for production of the scent cartridges 102 and the scent dispenser 100. Accordingly, embodiments are not limited to the ID formats or forms and associated hardware examples described above. For example, a physical shape of the scent cartridge 102 or portion thereof may encode information. For example, a shape of a profile or periphery (e.g., round, square, hexagonal) may indicate the type of scent cartridge 102 and hence provide some indication of the scents or types of scents carried by the scent cartridge 102. In reading the identification information carried by the ID structure, the reader 183 may determine the identity of a scent cartridge type, the identity of a particular scent cartridge 102 itself, the identity of each scent releasable from the scent cartridge 102, or the identity of each piece of scent media carried by the scent cartridge 102.

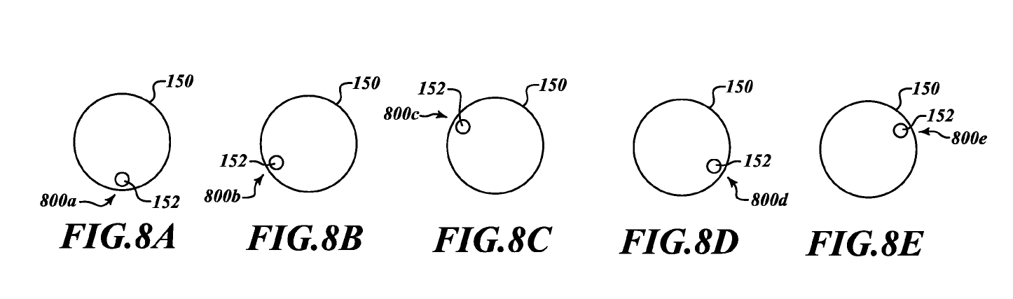

The contents indicator may include a picture, text, and/or a computer-readable structure (e.g., a barcode or RFID tag) indicating the particular scents, aromas, or other items contained in each of the chambers of the scent cartridge 102. For example, the contents indicator may indicate that a first specified passage includes scent media having a first scent (e.g., the scent of hazelnut coffee), a second specified passage includes scent media having a second scent (e.g., the scent of espresso), a third specified passage includes scent media having a third scent (e.g., the scent of Kona coffee), that a forth specified passage includes scent media that neutralize odors (e.g., baking soda), and that a fifth specified passage is empty. In one embodiment, the contents indicator is provided on a housing of the scent cartridge 102. FIG. 8A shows the baffle 150 in a first or default position 800a which, for example, aligns the port 152 with none of the passages 130 (not visible in FIG. 8A) of scent cartridge 102 in the respective scent cartridge receiver or receptacle 142 of the holder 122, thereby preventing dispensing (e.g., diffusion) of scent from the scent dispenser 100.

FIG. 8B shows the baffle 150 rotated or pivoted into a second position 800b which, for example, aligns the port 152 with a first one of the passages 130 of scent cartridge 102 in the respective scent cartridge receiver or receptacle 142 of the holder 122 (not visible in FIG. 8B) to dispense (e.g., via diffusion) scent of a first fragrance type from the scent dispenser 100.

FIG. 8C shows the baffle 150 rotated or pivoted into a third position 800c which, for example, aligns the port 152 with a second one of the passages 130 of scent cartridge 102 in the respective scent cartridge receiver or receptacle 142 of the holder 122 (not visible in FIG. 8C) to dispense (e.g., via diffusion) scent of a second fragrance type from the scent dispenser 100. The second fragrance type is preferably different from the first fragrance type, i.e., a different scent.

FIG. 8D shows the baffle 150 rotated or pivoted into a fourth position 800d which, for example, aligns the port 152 with a third one of the passages 130 of scent cartridge 102 in the respective scent cartridge receiver or receptacle 142 of the holder 122 (not visible in FIG. 8D) to dispense (e.g., via diffusion) scent of a third fragrance type from the scent dispenser 100. The third fragrance type is preferably different from the first and the second fragrance types, i.e., a different scent.

FIG. 8E shows the baffle 150 rotated or pivoted into a fifth position 800e which, for example, aligns the port 152 with a fourth one of the passages 130 of scent cartridge 102 in the respective scent cartridge receiver or receptacle 142 of the holder 122 (not visible in FIG. 8E) to dispense (e.g., via diffusion) scent of a fourth fragrance type from the scent dispenser 100. The fourth fragrance type is preferably different from the first, the second, and the third fragrance types, i.e., a different scent.

While not illustrated, the baffles can be positionable in fewer discrete positions (e.g., one, two or three positions), or can be positionable in a greater number of discrete positions (e.g., five or more positions). Hence, the scent dispenser 100 can be operable to dispense fewer or greater numbers of scents, each preferably of a different type or scent from one another, depending on the number of passages in the scent cartridges 102.

While not illustrated, the baffle 150 can include two ports 152, for example diametrically opposed from one another across the baffle 150. Such can, for example, provide additional surface area of the scent media to be concurrently aligned with respective ports 152, thereby allowing scent to be delivered in greater concentration. To achieve such, typically scent media of the same type would be loaded into diametrically opposed positions. Alternatively, pieces of scent media of different types could be loaded into diametrically opposed positions, allowing several combinations of scent to be dispensed.

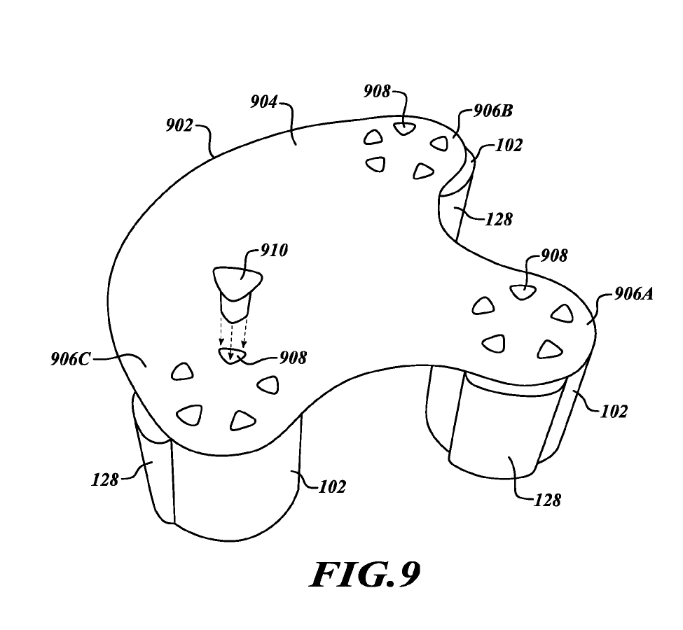

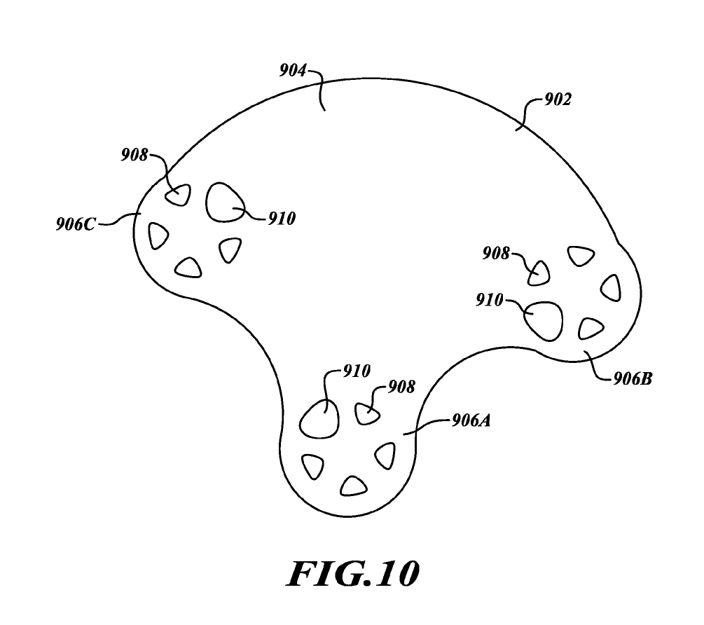

FIGS. 9 and 10 show an exemplary harness 902, according to at least one illustrated embodiment. Additionally or alternatively to the first cap cover 706, a scent media set may include the harness 902.

FIG. 9 shows the harness 902 physically coupled to hold a plurality of the scent cartridges 102 in a fixed position and orientation with respect to one another according to at least one illustrated implementation. The harness 902 and the scent cartridges 102 may be selectively loadable to and unloadable from the scent dispenser 100 as a single integral unit.

The harness 902 may be sized and dimensioned to permit a user to grip the harness 902 while the harness 902 holds the scent cartridges 102. The harness 902 may be sized and dimensioned to permit a user to haptically determine orientation of the scent cartridges 102 without touching the scent cartridges 102. For example, the harness may be sized and dimensioned to permit the user to haptically determine orientation of the scent cartridges 102 by gripping the harness without touching the scent cartridges 102. The harness 902 may comprise a plate 904. The plate 904 may be flat or substantially flat. The harness 902 may comprise a polymer. The harness 902 may have one or more lobes 906 for each of the plurality of scent cartridges 102. The harness may hold the scent cartridges 102 in fixed positions relative to each other at respective ones of the one or more lobes 906. For example, the harness may have a curvilinear profile with at least three lobes 906A-C, and each lobe holds one or more scent cartridges 102. The profile of the harness may be asymmetrical, thereby preventing the scent cartridges from being loaded in any position or orientation other than one position and orientation.

The harness 902 may have a plurality of through-holes 908 that extend therethrough. The number of the plurality of through-holes 908 may equal a number of respective passages 714 of the respective scent cartridges 102. The through-holes 908 may be in registration with the respective passages 714. The through-holes 908 may engage one or more of the respective passages 714 to securely fasten the respective scent cartridge 102 to the harness 908. For example, one or more of the through-holes 908 may engage one or more of the respective passages 714 in an interference fit to securely fasten the respective scent cartridge 102 to the harness 908.

Additionally or alternatively to one or more of the through-holes 908 securely fastening the respective scent cartridge 102 to the harness 902, at least one of the through-holes 908 may be sized and dimensioned to receive a respective plug 910. The plug 910 may comprise a polymer. The respective plug 910 may securely fasten one or more respective ones of the scent cartridges 102 to the harness 902. The one or more through-hole 908 may receive the respective plug 910 in an interference fit. Additionally or alternatively, one or more of the respective passages 714 of the one or more respective scent cartridge 102 may at least partially receive the plug 906 in an interference fit via the one or more through-hole 908 to securely fasten the one or more scent cartridge 102 to the harness 902. For example, the one or more through-hole 908 and the one or more respective passage 714 may receive the plug 906 in an interference fit to securely fasten the respective one or more scent cartridge 102 to the harness 902.

The harness 902 may define the fixed position and orientation of the respective one or more scent cartridge 102 relative to the other scent cartridges 102. The harness may define the fixed position and orientation in a manner that ensures that, when the harness 902 holds the plurality of the scent cartridges 102, the plurality of scent cartridges 102 and the harness 902 are selectively loadable to and unloadable from the scent dispenser 100 as a single integral unit. The harness 902 may define the fixed position and orientation in a manner that ensures that the keys 128 of the scent cartridges 102 concurrently align with the keyways 126 of the respective receptacles 124. The harness 902 may define the fixed position and orientation in a manner that ensures that a particular scent cartridge 102 is at a particular lobe 906 while another particular scent cartridge 102 is at another particular lobe 906. Such may permit the scent dispenser to determine all scent media contained in a scent media set that comprises the plurality of scent cartridges 102 by reading a single machine readable ID structure 185.

The one or more through-hole 908 may be sized, dimensioned, or positioned to receive the plug 910, and one or more other through-holes 908 may be sized, dimensioned, or positioned to preclude such from receiving the plug 910. Additionally or alternatively, the one or more through-hole 908 may be sized, dimensioned, or positioned to engage the one or more respective passage 714, and the one or more other through-holes 908 may be sized, dimensioned, or positioned to preclude such from engaging the one or more respective passage 714. Additionally or alternatively, the one or more through-hole 908 may be sized, dimensioned, or positioned to engage the one or more respective passage 714 in an interference fit to securely fasten the respective one or more scent cartridge 102 to the harness 902, and the one or more other through-holes 908 may be sized, dimensioned, or positioned to engage the one or more respective passage 714 yet are sized, dimensioned, or positioned to preclude such from engaging the one or more respective passage 714 in an interference fit to securely fasten the respective one or more scent cartridge 102 to the harness 902. Additionally or alternatively, the one or more through-hole 908 may be sized, dimensioned, or positioned to engage the one or more respective passage 714 only when each of the respective passages 714 are in registration with the respective through-holes 908.

Additionally or alternatively to the through-holes 908 being sized, dimensioned, or positioned to define the fixed position or orientation of the respective one or more scent cartridge 102, the one or more respective passage 714 may be sized, dimensioned, or positioned to receive the plug 910, and one or more other respective passages 714 may be sized, dimensioned, or positioned to preclude such from receiving the plug 910. Additionally or alternatively, the one or more other respective passages 714 may be sized, dimensioned, or positioned to receive the plug 910 yet sized, dimensioned, or positioned to preclude such from receiving the plug 910 in an interference fit to securely fasten the respective one or more scent cartridge 102 to the harness 902. Additionally or alternatively, the one or more respective passage 714 may be sized, dimensioned, or positioned to receive the plug 910 only when each of the respective passages 714 are in registration with the respective through-holes 908. The scent cartridges 102 are fixed and cannot rotate with respect to the harness 902 when secured to the harness 902 by respective ones of the plurality of plugs 906. For example, the plugs 906 may be positioned in through-holes that are offset or off axis from a longitudinal axis of the respective scent cartridge 102, preventing rotation of the scent cartridge 102.

### Operation

A reader 183 may read information from the various scent cartridges loaded in the scent cartridge receptacles. For example, the reader may read machine encoded information that unique identifies each scent cartridge, that identifies a scent cartridge by type or family, or that identifies the specific scents carried by respective scent cartridges and/or mapping the scents to particular passages or positions. The reading can be performed in response to a powering up of the scent dispenser, or on receipt of a new scent cartridge, and/or from time-to-time, either periodically or aperiodically.

The control subsystem 172 receives the read information. In some implementations, the control subsystem will used the read information (e.g., identifier) to retrieve information about the scent cartridge (e.g., scent mapping).

The control subsystem 172 can also receive information from the position encoders and assess a position of each of the baffles or ports. The control subsystem can, for example, initially return any baffles or ports to a first or default position to ensure no scent is being delivered or dispensed. The control subsystem achieves such by supplying signals to the corresponding motor controllers.

The control subsystem 172 can then cause the air mover (e.g., fan) to turn ON, drawing air into the interior of the housing via the inlets or vents.

The control subsystem 172 can then cause one or more baffles to move in order to emit or dispense selected scents, either individually or in combinations. The control subsystem achieves such by supplying signals to the corresponding motor controllers, which in turn cause the actuators (e.g., stepper motors) move the baffles to position the ports in various positions from a set of positions, including the above described first or default position. The control subsystem controls the amount or concentration of scent emitted or dispensed by controlling the time during which any given baffle is in a given position and/or fan speed.

For example, at least a first or default position prevents alignment with any passage 130, thereby preventing the dispersion or emission of scent when such is not desired. This can allow a period of time (e.g., approximately 30 seconds to 60 seconds) for a previously emitted scent to clear out or diminish in the ambient environment before another scent is dispersed from the scent dispenser 100. Alternatively, in some implementations, one or more passages 130 may be devoid of scent media, alignment with that passage 130 preventing the dispersion or emission of scent, and even allowing scent free air flow via the empty passage 130 to advantageously clear out any linger scents from the scent dispenser 100. Alternatively, one or more passages 130 can carry a neutral or neutralizing scent media (e.g., baking soda) that disperses or emits or an undiscernible scent or removes scent.

At least a second one of the positions provides air passage between a first one of the passages 130 of the scent cartridge 102 and the exterior 114 to allow scent to disperse from the first one of the passages 130 of the scent cartridge 102 via the port 152 to the exterior 114 while concurrently blocking air passage between at least a second one of the passages 130 of the scent cartridge 102 and the exterior 114. At least a third one of the positions provides air passage between the second one of the passages 130 of the scent cartridge 102 and the exterior via the port 152 to allow scent to disperse from the second one of the passages 130 of the scent cartridge 102 to the exterior 114 while concurrently blocking air passage between at least the first one of the passages 130 of the scent cartridge 102 and the exterior 114. As previously noted, the passages 130 of the scent cartridges 102 can carry scent media.

The scent dispenser can advantageously release of scent(s) in a vehicle to increase alertness while driving, and modulate the amounts and/or, types of scents released and/or times of release to avoids “nose fatigue” for the driver or pilot.

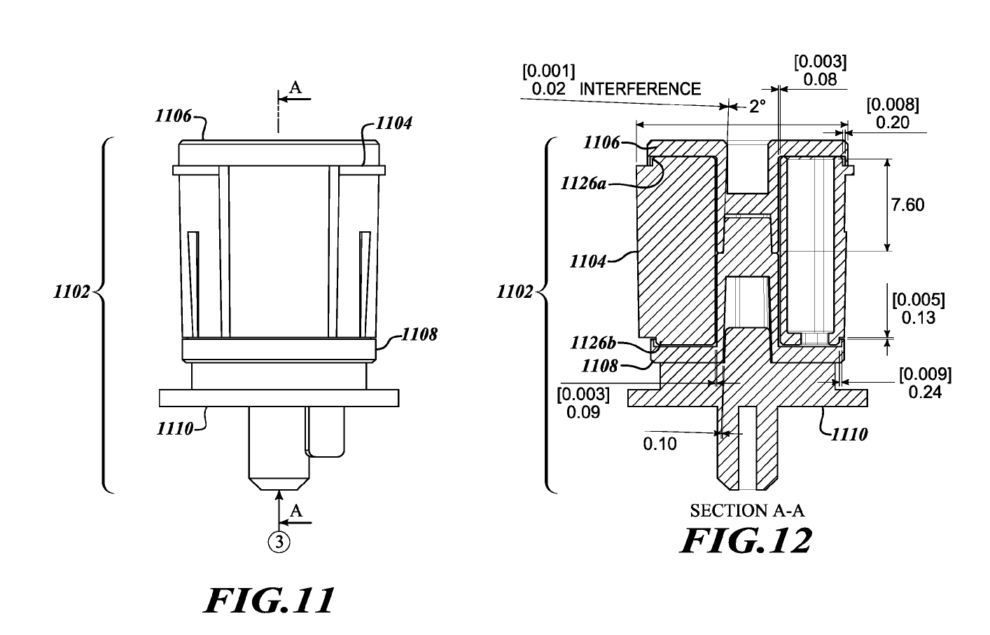

FIGS. 11 and 12 show a scent cartridge unit 1102 which includes a body 1104, a top cover or baffle 1106, a bottom cover or baffle 1108, and a drive interface 1110, coupled together as the integral scent cartridge unit 1102, according to at least one illustrated implementation.

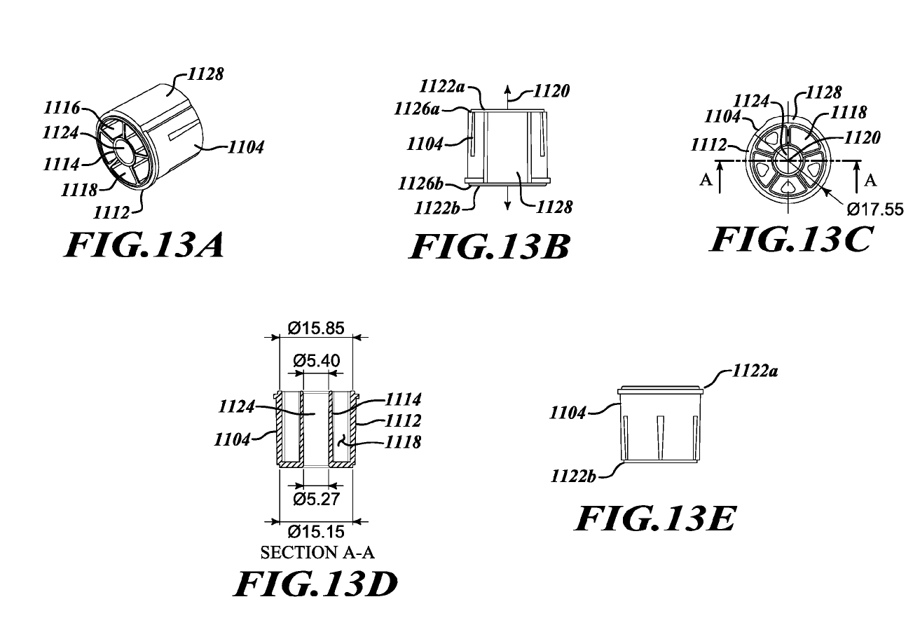

FIGS. 13A-13E show the body 1104 of the integral scent cartridge unit 1102 of FIG. 11 in more detail.

The body 1104 forms a housing, which may for example include an outer wall 1112 and an inner wall 1114 with a number webs 1116 (only one called out in FIG. 13A) extending therebetween to delineated a plurality of passages 1118 (only one called out in FIGS. 13A, 13C, 13D) that axially extend through the body 1104, the passages 1118 arrayed about a central axis (FIGS. 13B and 13C) of the body 1104. The outer wall 1112 may be tubular, for instance an outer cylindrical tube, and the inner wall 1114 may be tubular, for instance an inner cylindrical tube. The body 1104 is generally open at a first or top end 1122a (FIGS. 13A, 13E) and at a second or bottom end 1122b (FIGS. 13A, 13E).

The passages 1118 may, for example, have a wedge shape or piece of pie shape profile. Each of the passages 1118 can contain scent media. For example, each passage 1118 can contain a respective quantity of scent media that emits a respective one of a plurality of scents. One or more passage 1118 can be devoid of scent media, and optionally can contain a neutralizing media (e.g., activated carbon, baking soda).

The body 1104 may include a central passage or recess 1124 (FIGS. 13A, 13C, 13D) formed by the inner wall 1114. The central passage or recess 1124 is sized and dimensioned to closely receive a portion of the top cover or baffle 1106 and a portion of the bottom cover or baffle 1108. While the central passage or recess 1124 closely receives the portions of the top cover or baffle 1106 and the bottom cover or baffle 1108, the top cover or baffle 1106 and the bottom cover or baffle 1108 may be spaced from the body 1104 by a gap, for example a gap of about 0.003 inches (e.g., plus or minus 10%). Such can reduce unintentional dispersion of scent, while allowing relative movement between body 1104 and both the top cover or baffle 1106 and the bottom cover or baffle 1108.

The body 1104 may include an upper peripheral lip or edge 1126a (FIGS. 12 and 13A) and a lower peripheral lip or edge 1126b that (FIGS. 12 and 13A) provides an edge to mate with or be received by corresponding portions of the top cover or baffle 1106 and the bottom cover or baffle 1108, respectively. The body 1104 may include key 1128 (FIGS. 13A, 13B, 13C) that extends laterally outward relative to the central axis 1120.

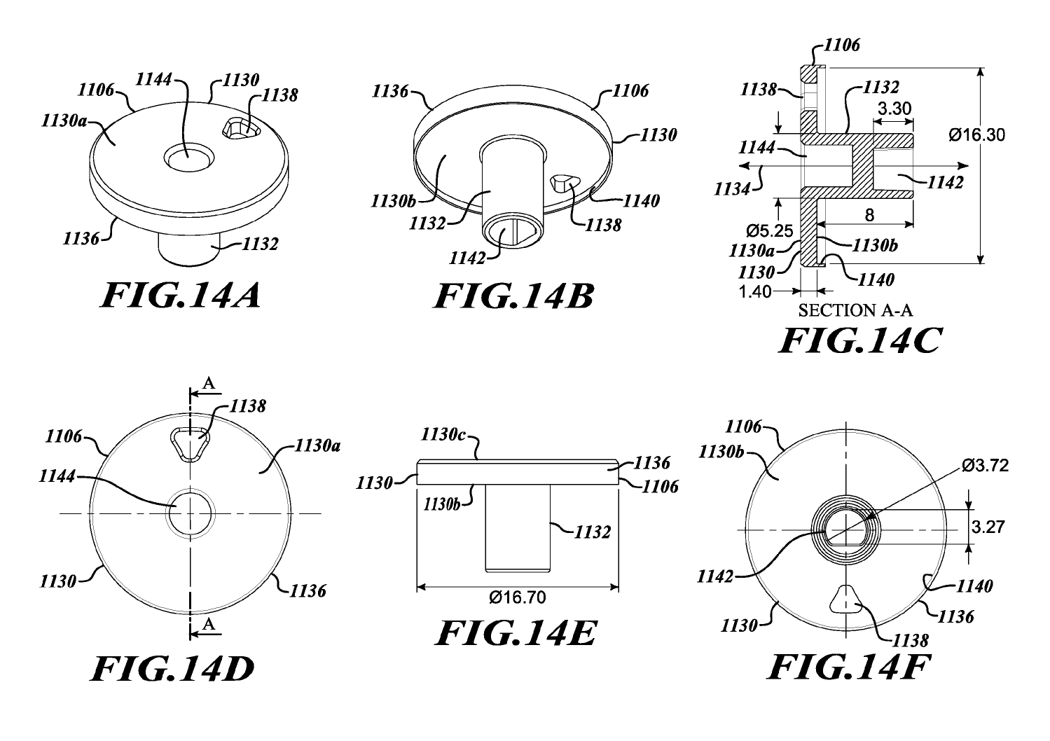

FIGS. 14A-14F show the top cover or baffle 1106 of the integral scent cartridge unit 1102 of FIG. 11 in more detail.

The top cover or baffle 1106 includes a disc 1130 with an upper surface 1130a and a lower surface 1130b, and includes a stem 1132 that extends downward from the lower surface 1130b of the disc 1130. The disc 1130 may have a longitudinal axis 1134 (FIG. 14C) and circular profile with a circular peripheral edge 1136. The stem 1132 may be aligned with the longitudinal axis 1134. The disc 1130 includes an aperture 1138 that extends therethrough. The aperture 1138 may be sized and dimensioned to match the profile of the passages 1118 of the body 1104. The disc 1130 may include a lip, for example a peripheral lip 1140 that extends downward from the lower surface 1130b (FIGS. 14B, 14C, 14D). The peripheral lip 1140 may be sized and dimensioned to mate with or receive the upper peripheral lip 1126a (FIGS. 12 and 13B) of the body 1104.

The stem 1132 includes a lower aperture 1142 (FIGS. 14B, 14C, 14F) accessible from the lower surface 1130b of the disc 1130. The lower aperture 1142 may extend along the longitudinal axis 1134 and is sized and dimension to securely fit (e.g., interference fit) with a corresponding or complementary portion of the bottom cover or baffle 1108. The lower aperture 1142 can, for example, have a D-shaped profile or other non-circular profile (e.g., square, hexagonal) to facilitate transmission of torque from the bottom cover or baffle 1106 to the top cover or baffle 1106. The stem 1132 and/or disk 1130 may include an upper aperture 1144 accessible from the upper surface 1130a of the disc 1130.

FIGS. 15A-15D show the bottom cover or baffle 1106 of the integral scent cartridge unit 1102 of FIG. 11 in more detail.

The bottom cover or baffle 1108 includes a disc 1146 with an upper surface 1146a and a lower surface 1146b, and includes a stem 1148 that extends upward from the upper surface 1146a of the disc 1146. The disc 1146 may have a longitudinal axis 1150 (FIG. 15D) and circular profile with a circular peripheral edge 1152. The stem 1148 may be aligned with the longitudinal axis 1150. The disc 1146 includes an aperture 1153 (FIGS. 15A, 15C) that extends therethrough. The aperture 1153 may be sized and dimensioned to match the profile of the passages 1118 of the body 1104. The disc 1146 may include a lip, for example a peripheral lip 1154 (FIGS. 15C and 15D) that extends upward from the upper surface 1146a. The peripheral lip 1154 may be sized and dimensioned to mate with or receive the lower peripheral lip 1126b (FIGS. 12 and 13B) of the body 1104.

The stem 1148 includes a tapered portion 1148a (FIGS. 15B and 15D) at a distal end 1156 of the stem 1148, the tapered portion 1148a having an increasingly smaller periphery as an upper portion of the stem 1148 is traversed toward the distal most end 1156 of the stem 1148. The tapered portion 1148a may extend along the longitudinal axis 1150 and is sized and dimension to securely fit (e.g., interference fit) with a corresponding or complementary portion (e.g., in lower aperture) of the upper cover or baffle 1106. The tapered portion 1148a can, for example, have a D-shaped profile or other non-circular profile (e.g., square, hexagonal) to facilitate transmission of torque from the bottom cover or baffle 1106 to the top cover or baffle 1106. The stem 1148 and/or disk 1146 may include a lower aperture 1158 (FIGS. 15C, 15D) accessible from the lower surface 1146b of the disc 1146. The lower aperture 1158 can be sized and dimensioned to cooperate engage a corresponding or complimentary portion on the drive interface 1110.

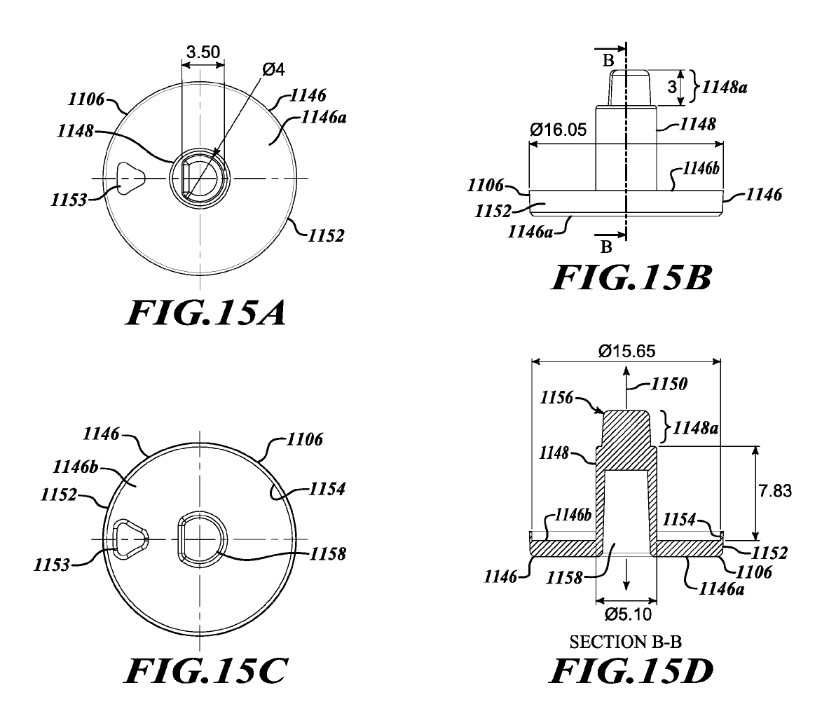

FIGS. 16A-16F show the drive interface 1110 of the integral scent cartridge unit 1102 of FIG. 11.

The drive interface 1110 includes a disc 1160 with an upper surface 1160a and a lower surface 1160b, and includes a upper stem 1162 that extends upward from the upper surface 1160a of the disc 1160 and a lower stem 1164 that extends downwardly from the lower surface 1160b of the disc 1160. The disc 1160 may have a longitudinal axis 1166 and circular profile with a circular peripheral edge 1168. The stems 1162, 1164 may be aligned with the longitudinal axis 1166. The disc 1160 includes an aperture 1169 (FIGS. 16A, 16B, 16C, 16D and 16F) that extends therethrough. The aperture 1169 may be sized and dimensioned to match the profile of the passages 1118 of the body 1104. The aperture 1169 of the drive interface 1110 may advantageously align with the aperture 1153 of the bottom cover or baffle 1108 and the aperture 1138 of the top cover or baffle 1106, all of which are selectively alienable with respective ones of the passages 1118 of the body 1104 to selectively emit or disperse one scent while preventing or limiting emission or dispersion of other scents.

The upper stem 1162 may be sized and dimensioned to match the profile of the lower aperture 1158 (FIG. 15D) of the bottom cover or baffle 1108. The upper stem 1162 may, for example, optionally include a tapered portion, the tapered portion having an increasingly smaller periphery as the upper stem 1162 is traversed toward the distal most end of the upper stem 1162. The tapered portion may extend along the longitudinal axis 1166 and is sized and dimension to securely fit (e.g., interference fit) with a corresponding or complementary portion (e.g., in lower aperture 1158) of the lower cover or baffle 1108. The tapered portion can, for example, have a D-shaped profile or other non-circular profile (e.g., square, hexagonal) to facilitate transmission of torque from the drive interface to the bottom cover or baffle 1108. The lower stem 1164 may be sized and dimensioned to mate with or a drive shaft of a motor of a scent dispenser into which the integral scent cartridge unit 1102 is removably loaded. For example, the lower stem 1164 may include an aperture 1168 sized and dimensioned to securely receive a drive shaft of a motor. The aperture may have a D-shaped profile or other non-circular profile to facilitate transmission of torque from the motor to the drive interface. Alternatively or additionally, a key 1170 (FIGS. 16B, 16D, 16E, 16F) may extend from the disc 1160, for example upwardly from the upper surface 1160a to facilitate transmission of torque from the motor to the drive interface. The drive interface 1110 may be comprises of a polymer, for example LEXAN® 940A or similar material.

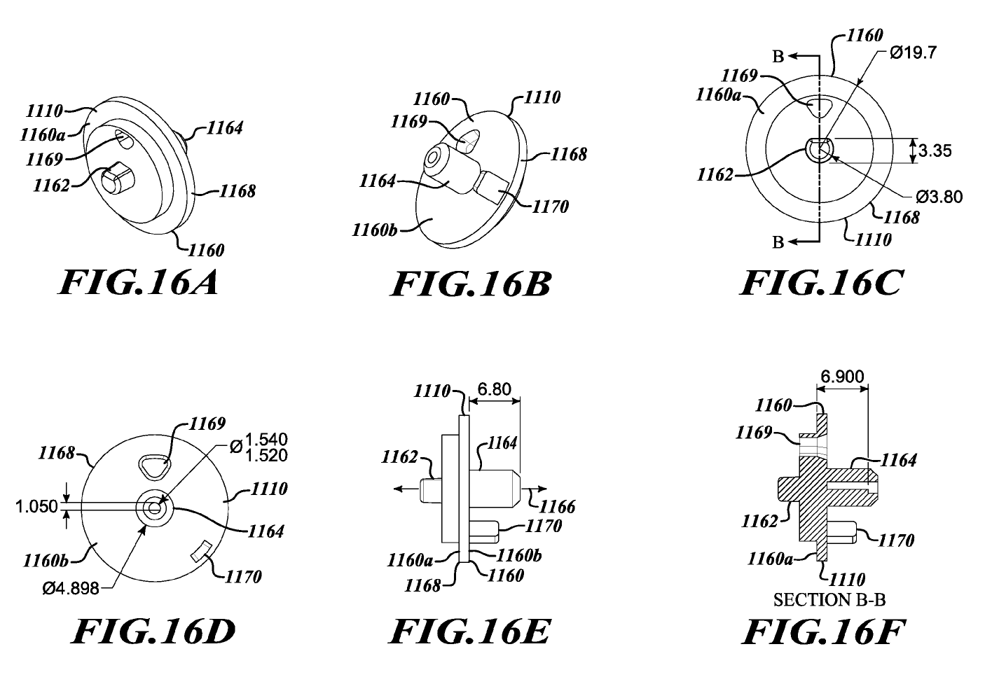

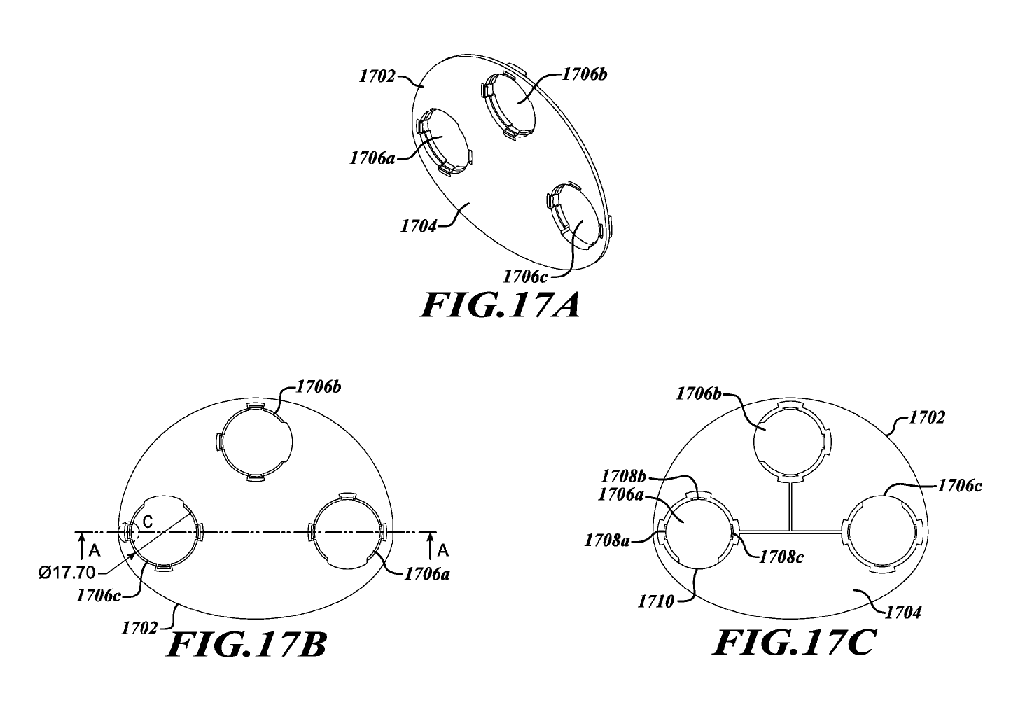

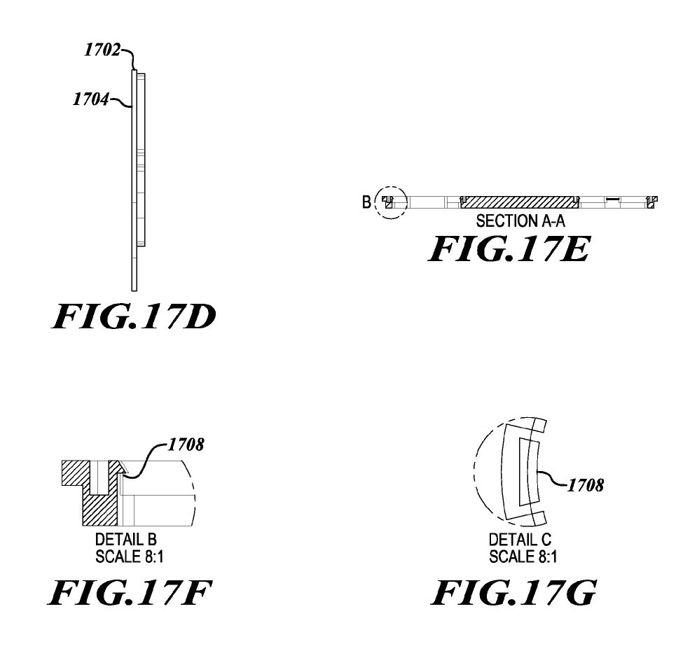

FIGS. 17A-17G show a harness 1702 for retaining three of the scent cartridge units of FIG. 11, according to at least one illustrated implementation.

The harness 1702 includes a substrate 1704 having a plurality of openings 1706a, 1706b, 1706c (three showing, collectively 1706), the openings 1706 each sized and dimensioned to securely receive portions of respective ones of the scent cartridge units therethrough. Each of the openings 1706 may include a plurality of detents 1708a, 1708b, 1708c (three shown for a single opening 1706a) to retain the scent cartridge units and a keyway 1710 (one shown for a single opening 1706a) to secure the scent cartridge units against rotation.

The above description of illustrated implementations, including what is described in the Abstract, is not intended to be exhaustive or to limit the implementations to the precise forms disclosed. Although specific implementations of and examples are described herein for illustrative purposes, various equivalent modifications can be made without departing from the spirit and scope of the disclosure, as will be recognized by those skilled in the relevant art. The teachings provided herein of the various implementations can be applied to other systems, not necessarily the exemplary systems generally described above.

The foregoing detailed description has set forth various implementations of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one implementation, the present subject matter may be implemented via application-specific integrated circuits (ASICs). However, those skilled in the art will recognize that the implementations disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

Those of skill in the art will recognize that many of the methods or algorithms set out herein may employ additional acts, may omit some acts, and/or may execute acts in a different order than specified.

In addition, those skilled in the art will appreciate that the mechanisms taught herein are capable of being distributed as a program product in a variety of forms, and that an illustrative implementation applies equally regardless of the particular type of signal bearing media used to actually carry out the distribution. Examples of signal bearing media include, but are not limited to, the following: recordable type media such as floppy disks, hard disk drives, CD ROMs, digital tape, and computer memory.

The various implementations described above can be combined to provide further implementations. To the extent that they are not inconsistent with the specific teachings and definitions herein, all of the U.S. patents, U.S. patent application publications, U.S. patent applications, foreign patents, foreign patent applications and non-patent publications referred to in this specification, including U.S. Patent Application Ser. Nos. 61/792,716, filed Mar. 15, 2013; 61/817,180, filed Apr. 29, 2013; 61/822,270, filed May 10, 2013; 61/891,328, filed Oct. 15, 2013; Ser. No. 14/213,608, filed Mar. 14, 2014; Ser. No. 14/213,683, filed Mar. 14, 2014; 61/944,862, filed Feb. 26, 2014; 62/069,104, filed Oct. 27, 2014; 61/944,866, filed Feb. 26, 2014; 62/012,863, filed Jun. 16, 2014; 62/116,258, filed Feb. 13, 2015; 61/944,870, filed Feb. 26, 2014; 62/199,763, filed Jul. 31, 2015; 62/237,304, filed Oct. 5, 2015; 62/314,727 filed Mar. 29, 2016; and 62/398,994, filed Sep. 23, 2016, are incorporated herein by reference, in their entirety. Aspects of the implementations can be modified, if necessary, to employ systems, circuits and concepts of the various patents, applications and publications to provide yet further implementations.

These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the claims are not limited by the disclosure.

1. A scent dispenser, comprising:
a housing having a first end, a second end opposed to the first end, an interior, and a plurality of vents that allow a passage of air to the interior of the housing from an exterior thereof;
a holder coupled to the housing, the holder having at least one scent cartridge receiver to removably hold a respective scent cartridge, the scent cartridge having a plurality of passages, at least one of which contains respective scent media;
at least one baffle having a port, the baffle mounted for movement with respect to the at least one scent cartridge receiver between a plurality of positions, at least a first one of the positions which provides air passage between a first one of the passages of the scent cartridge and the exterior to allow scent via the port to disperse from the first one of the passages of the scent cartridge to the exterior while concurrently blocking air passage between at least a second one of the passages of the scent cartridge and the exterior, and at least a second one of the positions which provides air passage between the second one of the passages of the scent cartridge and the exterior via the port to allow scent to disperse from the second one of the passages of the scent cartridge to the exterior while concurrently blocking air passage between at least the first one of the passages of the scent cartridge and the exterior;
at least one actuator selectively operable to move the baffle with respect to the at least one scent cartridge receiver between the plurality of positions; and
a control subsystem comprising circuitry communicatively coupled and operable to control the at least one actuator to selectively move the at least one baffle with respect to the at least one scent cartridge receiver between the plurality of positions.

2. The scent dispenser of claim 1 wherein the at least one actuator comprises an electric stepper motor having a drive shaft, the electric stepper motor physically coupled in the housing and the drive shaft drivingly coupled to the at least one baffle.

3. The scent dispenser of any of claim 1 or 2 wherein the at least one baffle is a circular plate and the drive shaft of the at least one actuator is drivingly coupled to rotate the at least one baffle.

4. The scent dispenser of claim 3 wherein the control subsystem is operable cause the electric stepper motor to rotate the at least one baffle in a clockwise rotation at one time and in a counterclockwise rotation at another time.

5. The scent dispenser of claim 4 wherein the at least one scent cartridge receiver comprises at least one receptacle sized and dimensioned to hold a respective scent cartridge, the at least one receptacle having a keyway, the keyway sized and dimensioned to securely receive a key on the respective scent cartridge to restrain the respective scent cartridge from rotation.

6. The scent dispenser of claim 4, further comprising:
at least one spring, the at least one spring positioned to bias the at least one baffle into contact with a portion of the scent media cartridge.

7. The scent dispenser of claim 6 wherein the at least one spring is positioned between a portion of the electric stepper motor and the at least one baffle.

8. The scent dispenser of any of claim 1 or 2 wherein the vents are positioned at least proximate a second end of the housing, further comprising:
a plurality of substrates received in the housing between the first and the second ends of the housing, each of the substrates having passage therethrough that provides a flow of air between the vents and the first end of the housing.

9. The scent dispenser of claim 8 wherein the vents are positioned at least proximate the second end of the housing, further comprising:
an air mover received in the housing and communicatively coupled to the control subsystem for control thereby, the air mover operable to actively cause the flow of air between the vents and the first end of the housing.

10. The scent dispenser of claim 9, further comprising:
a grille removably coupled to the housing proximate the first end of the housing, the grille removable from the housing to access the at least one scent cartridge receiver.

11. The scent dispenser of claim 10, further comprising:
a shroud in which the grille is mounted and which removably couples the grille to the housing, wherein the shroud is metal and the housing is plastic.

12. The scent dispenser of any of claim 1 or 2 wherein the control subsystem comprises at least one wired or wireless communications port or receiver.

13. The scent dispenser of claim 1 wherein the plurality of vents are at least proximate the second end of the housing and the housing includes a plurality of spacers in the interior thereof, and further comprising:
a battery received in the housing and spaced from the vents by the spacers.

14. The scent dispenser of claim 1 wherein:
the holder has at least three scent cartridge receivers to removably hold respective scent cartridges;
the at least one baffle comprises a respective baffle having a respective port for each of the scent cartridge receivers of the holder, the baffles mounted for rotational movement with respect to a respective one of the scent cartridge receivers between a plurality of positions, at least a first one of the positions which provides air passage between a first one of the passages of the scent cartridge and the exterior to allow scent via the port to disperse from the first one of the passages of the scent cartridge to the exterior while concurrently blocking air passage between at least a second one of the passages of the scent cartridge and the exterior, and at least a second one of the positions which provides air passage between the second one of the passages of the scent cartridge and the exterior via the port to allow scent to disperse from the second one of the passages of the scent cartridge to the exterior while concurrently blocking air passage between at least the first one of the passages of the scent cartridge and the exterior;
the at least one actuator comprises one stepper motor for each of the baffles, the stepper motors drivingly coupled and selectively operable to move the respective baffle with respect to the respective scent cartridge receiver between the plurality of positions; and
the control subsystem is operable to control the actuators to selectively move the respective baffles with respect to the respective scent cartridge receivers between the plurality of positions.

15. The scent dispenser of claim 14 wherein the holder has at least one lip that closely receives the baffles.

16. The scent dispenser of any of claim 14 or 15, further comprising:
a plurality of scent cartridges, each of the scent cartridges removably held by a respective one of the scent cartridge receivers of the holder, each of the scent cartridges having a respective first end, a respective second end, a respective plurality of passages that extend between the first and the second ends, the passages circumferentially distributed about an axis of the cartridge and spaced radially from the axis of the cartridge, and at least two of the passages contain respective scent media, the scent media in a first one passages having a first scent and the scent media in a second one passages having a second scent, the second scent different from the first scent.

17. The scent dispenser of claim 14 wherein the vents are positioned at least proximate a second end of the housing, further comprising:
a first substrate received in the housing, the first substrate having a passage therethrough, the first substrate positioned between the holder and the second end of the housing;
a second substrate received in the housing, the second substrate having a passage therethrough, the second substrate positioned between the first substrate and the second end of the housing; and
a third substrate received in the housing, the third substrate having a passage therethrough, the third substrate positioned between the second substrate and the second end of the housing; the passages in the first, the second, and the third substrates providing a flow of air between the vents and the first end of the housing.

18. The scent dispenser of claim 17 wherein the housing is cylindrical and the first, the second, and the third substrates are annular printed circuit boards, and wherein the stepper motors are carried by the first substrate, spaced between the first and the second substrates.

19. The scent dispenser of claim 18 wherein the control subsystem is carried by the third substrate.

20. The scent dispenser of claim 19, further comprising:
an air mover received in the housing and carried by the second substrate, the air mover positioned axially in line with the passage of the second substrate.

21. The scent dispenser of claim 20 wherein the plurality of vents are at least proximate the second end of the housing and the housing includes a plurality of spacers in the interior thereof, and further comprising:
a battery received in the housing and spaced from the vents by the spacers.

22. The scent dispenser of claim 21, further comprising:
a grille removably coupled to the housing proximate the first end of the housing, wherein the plurality of vents are at least proximate the second end of the housing.

23. The scent dispenser of any of claim 14 or 15 wherein the plurality of scent cartridge receivers each comprise a respective receptacles sized and dimensioned to hold respective ones of the plurality of scent cartridges and restrain the respective scent cartridges from rotation.

24. The scent dispenser of claim 23 wherein each of the receptacles has a keyway, the keyways sized and dimensioned to securely receive a key on each of the scent cartridges to restrain the respective scent cartridges from rotation.

25. The scent dispenser of claim 23 wherein each of the baffles is mounted for movement with respect to a respective one of the scent cartridge receivers between at least three positions, a first one of the positions which provides communicative coupling between a first one of the passages of the respective scent cartridge mounted in the respective scent cartridge receiver and the port of the respective baffle, and a second one of the positions which provides communicative coupling between a second one of the passages of the respective scent cartridge mounted in the respective scent cartridge receiver and the port of the respective baffle.

26. The scent dispenser of claim 25 wherein a third one of the positions provides communicative coupling between a third one of the passages of the respective scent cartridge mounted in the respective scent cartridge receiver and the port of the respective baffle.

27. The scent dispenser of claim 25 wherein a third one of the positions provides communicative coupling between none of the channels of the respective scent cartridge and the port of the respective baffle, to prevent dispersion of scent from the interior of the scent dispenser to the exterior thereof.

28. The scent dispenser of any of claim 1 or 14 wherein the housing has a diameter of approximately 3¼ inch to 3¾ inch.

29. A method of operation of a scent dispenser, the scent dispenser comprising a housing; a holder received in the interior of the housing, the holder having a number of scent cartridge receivers to removably hold respective scent cartridges, the scent cartridges having a plurality of passages at least one of which contains respective scent media; a number of baffles having a port; a number of actuators selectively operable to move a respective one of the baffles with respect to the at least one scent cartridge receiver between a plurality of positions, the method comprising:
driving a first one of the baffles to a first one of the positions in which the port of the first one of the baffles is aligned with a first one of the passages of a first one of the scent cartridges which is mounted in a first one of the respective scent cartridge receivers; and
driving the first one of the baffles to a second one of the positions in which the port of the first one of the baffles is not aligned with the first one of the passages of the first one of the scent cartridges which is mounted in the first one of the respective scent cartridge receivers.

30. The method of claim 29 wherein driving the first one of the baffles to a second one of the positions includes driving the first one of the baffles to the second one of the positions in which the port of the first one of the baffles is aligned with a second one of the passages of the first one of the scent cartridges which is mounted in the first one of the respective scent cartridge receiver.

31. The method of claim 29 wherein driving the first one of the baffles to a second one of the positions includes driving the first one of the baffles to the second one of the positions in which the port of the first one of the baffles is aligned with a second one of the passages of the first one of the scent cartridges which is mounted in the first one of the respective scent cartridge receiver, the second one of the passages devoid of scent media.

32. The method of claim 29 wherein driving the first one of the baffles to a second one of the positions includes driving the first one of the baffles to the second one of the positions in which the port of the first one of the baffles is aligned with a second one of the passages of the first one of the scent cartridges which is mounted in the first one of the respective scent cartridge receiver, the second one of the passages containing a neutral scent media.

33. The method of claim 29, further comprising:
driving a second one of the baffles to a first one of the positions in which the port of the second one of the baffles is aligned with a first one of the passages of a second one of the scent cartridges which is mounted in a second one of the respective scent cartridge receivers.

34. The method of claim 33 wherein driving a second one of the baffles to a first one of the positions is performed concurrently with driving the first one of the baffles to the first one of the positions.

35. The method of claim 33 wherein driving a second one of the baffles to a first one of the positions is performed concurrently with driving the first one of the baffles to the second one of the positions.

36. The method of claim 33 wherein driving a second one of the baffles to a first one of the positions is performed after driving the first one of the baffles to the first one of the positions.

37. The method of claim 33 wherein driving a second one of the baffles to a first one of the positions is performed after driving the first one of the baffles to the second one of the positions.

38. The method of claim 29, further comprising:
driving a third one of the baffles to a first one of the positions in which the port of the third one of the baffles is aligned with a first one of the passages of a third one of the scent cartridges which is mounted in a third one of the respective scent cartridge receivers.

39. The method of claim 29 wherein the scent dispenser further comprises a fan received in the housing, further comprising:
driving the fan to create an air flow through the housing from a plurality of vents at least proximate a bottom of the housing to an exist proximate a top of the housing.

40. The method of claim 29, further comprising:
receiving signals indicative of a sequence of scents to be delivered from the housing; and
driving the number of baffles responsive to the received signals.

41. A scent media set useable with a scent dispenser, the scent media set, comprising:
a plurality of scent cartridges, each of the scent cartridges having a body having a first end and a second end, the second end opposed to the first end, each of bodies of the scent cartridges respectively having a plurality of passages that extend through the body with respective ports at the first and the second ends, at least one of the passages of each of the plurality of passages which contains a respective scent media; and
a harness physically coupled to hold the plurality of scent cartridges in a fixed position and orientation with respect to one another.

42. The scent media set of claim 41 wherein the plurality of scent cartridges includes at least three scent cartridges.

43. The scent media set of any of claim 41 or 42 wherein each body includes at least three passages, and at least two of the passages of each body contain a respective scent media.

44. The scent media set of claim 43 wherein at least a first passage of at least one body contains a first scent media that disperses a first scent, and at least a second passage of at least one body contains a second scent media that disperses a second scent, the second scent different from the first scent.

45. The scent media set of claim 43 wherein at least one passage of at least one body contains no scent media.

46. The scent media set of claim 43 wherein at least one passage of at least one body contains a neutral scent media that is non-discernable as a specific scent by humans.

47. The scent media set of any of claim 41 or 42 wherein each body includes five passages, and four of the passages of each body contain respective scent media and a fifth one of the passages of each body contains no scent media.

48. The scent media set of any of claim 41 or 42 wherein the plurality of scent cartridges and the harness are selectively loadable to and unloadable from the scent dispenser as a single integral unit.

49. The scent media set of any of claim 41 or 42 wherein the harness has a plurality of through-holes that extend therethrough, a first set of through-holes of the plurality of through-holes are in registration with respective ones of a number of the passages of a respective first one of the scent cartridges; a second set of through-holes of the plurality of through-holes are in registration with respective ones of a number of the passages of a respective second one of the scent cartridges.

50. The scent media set of claim 49 wherein a third set of through-holes of the plurality of through-holes are in registration with respective ones of a number of the passages of a respective third one of the scent cartridges.

51. The scent media set of claim 50 wherein each body includes five passages, and four of the passages of each body contain respective scent media and a fifth one of the passages of each body contains no scent media.

52. The scent media set of claim 49 further comprising:
a plurality of plugs, for each of the scent cartridges at least one respective plug received through a respective one of the through-holes and at least partially received in a respective one of the passages to securely fasten the respective scent cartridge to the harness.

53. The scent media set of claim 52 wherein the plugs are received in an interference fit with the respective through-hole and the respective passage to securely fasten the respective scent cartridge to the harness.

54. The scent media set of claim 52 wherein the scent cartridges are fixed and cannot rotate with respect to the harness when secured to the harness by respective ones of the plurality of plugs.

55. The scent media set of claim 52 wherein the harness and the plugs each comprise a polymer.

56. The scent media set of claim 49 wherein the harness comprises a polymer.

57. The scent media set of claim 49 wherein the harness comprises a flat plate of a polymer.

58. The scent media set of claim 57 wherein the flat plate has a curvilinear profile with three lobes, each lobe delimiting a location of a respective one of the scent cartridges.
